SEQUENCE LISTING (6 sequences)

SEQ ID NO: 1 - DNA, 24 bp, Artificial
  synthetic PCR primer
ttcaagacctggcccagtgcatcc

SEQ ID NO: 2 - DNA, 24 bp, Artificial
  synthetic PCR primer
agcaacaaccctgccctgtgcaac

SEQ ID NO: 3 - DNA, 20 bp, Artificial
  synthetic PCR primer
cacttcaaccacagtggcat

SEQ ID NO: 4 - DNA, 20 bp, Artificial
  synthetic PCR primer
attcacatactccctgggga

SEQ ID NO: 5 - DNA, 20 bp, Artificial
  synthetic PCR primer
gagaacgggaagcttgtcat

SEQ ID NO: 6 - DNA, 20 bp, Artificial
  synthetic PCR primer
ttcagctcagggatgacctt

(19) United States
(12) Patent Application Publication
(10) Pub. No.: US 2009/0325877 A1
(43) Pub. Date: Dec. 31, 2009

(54) Combination Product of Receptor Tyrosine Kinase Inhibitor and Fatty Acid Synthase Inhibitor for Treating Cancer

(75) Inventors: Thomas Grunt, Baden (AT); Renate Wagner, Vienna (AT)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/470,613

(22) Filed: May 22, 2009

Related U.S. Application Data
(60) Provisional application No. 61/056,015, filed on May 25, 2008.
(60) Provisional application No. 61/117,367, filed on Nov. 24, 2008.

(51) Int. Cl. A61K 38/16; C12N 5/06; A61K 31/47; A61K 31/4709; A61P 35/00

(52) U.S. Cl. 514/12; 435/325; 514/311; 514/314

(57) ABSTRACT

A pharmaceutical combination product is disclosed that comprises a receptor tyrosine kinase inhibitor and a fatty acid synthase inhibitor, and to the use thereof in the manufacture of a medicament for use in the treatment or prophylaxis of cancer.

### CROSS-REFERENCE TO RELATED APPLICATIONS

This application claims the benefit under 35 U.S.C. §119(e) to co-pending U.S. Provisional Application Ser. No. 61/056,015, filed May 25, 2008 and U.S. Provisional Application Ser. No. 61/117,367, filed Nov. 24, 2008, which are hereby incorporated by reference in their entirety.

### FIELD OF THE INVENTION

The present invention relates to a combination comprising an inhibitor of the receptor tyrosine kinase family, or a pharmaceutically acceptable salt thereof, and a fatty acid synthase inhibitor. In particular, the combination product is directed to certain 4-anilino-3-cyanoquinolines or a pharmaceutically acceptable salt thereof, and a fatty acid synthase inhibitor. The combination product of the invention is useful in a new method for the treatment or prophylaxis of cancer. The invention also relates to a pharmaceutical composition comprising such a combination product and to the use thereof in the manufacture of a medicament for use in the treatment or prophylaxis of cancer.

### BACKGROUND OF THE INVENTION

Tyrosine kinases (TKs) are divided into two classes: the non-transmembrane TKs and transmembrane growth factor receptor TKs (RTKs) as described by Blume-Jensen, P., Nature, 411, 355 (2001). Growth factors, such as epidermal growth factor (EGF), bind to the extracellular domain of their partner RTK on the cell surface, which activates the RTK, initiating a signal transduction cascade that controls a wide variety of cellular responses including proliferation and migration. The overexpression of EGF and also of members of the epidermal growth factor receptor (EGFR) family, which includes epidermal growth factor receptor (EGFR, ErbB1) ErbB2 (HER-2 neu), ErbB3 and ErbB4, is implicated in the development and progression of cancer, as described by Rusch, V., Cytokine Growth Factor Rev., 7, 133 (1996), Davies, D. E., Biochem. Pharmacol., 51, 1101 (1996) and Modjtahedi, E., Int. J. Oncol., 4, 277 (1994). Specifically, over expression of the receptor kinase product of the erbB-2 oncogene has been associated with human breast and ovarian cancers, as described by Slamon, D. J., Science, 244, 707 (1989) and Slamon, D. J., Science, 235, 177 (1987). Upregulation of EGFR kinase activity has been associated with epidermoid tumors, as described by Reiss, M., Cancer Res., 51, 6254 (1991)]; breast tumors, as described by Macias, A., Anticancer Res., 7, 459 (1987); and tumors involving other major organs, as described by Gullick, W. J., Brit. Med. Bull., 47, 87 (1991). Certain 3-cyanoquinolines are irreversible inhibitors of RTK, including the EGFR family and have exhibited anti-tumor activity, as described by Rabindran et al, Cancer Research, 64, 3958-3965 (2004).

Fatty acid synthase (FASN or FAS) catalyzes the conversion of acetyl CoA and malonyl-CoA, in the presence of NADPH, into long-chain saturated fatty acids, as described by Wakil, Biochemistry 28:4523-4530 (1989). In prokaryotes and plants, FASN consists of an acyl carrier protein and 7 structurally independent monofunctional enzymes. In animals, however, all of the component enzymatic activities of FASN and acyl carrier protein are organized in one large polypeptide chain. Loftus et al., in Science 288:2379-2381 (2000), identified a link between anabolic energy metabolism and appetite control. Both systemic and intracerebroventricular treatment of mice with FASN inhibitors (cerulenin and C75, a synthetic compound), led to inhibition of feeding and dramatic weight loss. The compound C75 inhibited expression of the prophagic signal neuropeptide Y in the hypothalamus and acted in a leptin-independent manner that appears to be mediated by malonyl-CoA. Loftus et al. further suggested that FASN may represent an important link in feeding regulation and may be a potential therapeutic target for obesity.

Treatment of ovarian cancer (OC) is still suboptimal, necessitating the search for novel therapies. In normal tissue, the key lipogenic enzyme fatty acid synthase (FASN) converts dietary carbohydrates to triglycerides, whereas in cancer, FASN represents a metabolic oncogene and produces phospholipids for membrane microdomains (lipid rafts) that accommodate clusters of receptor tyrosine kinases including Epidermal Growth Factor Receptor (EGFR, ErbB1) and ErbB2 (HER-2/neu), thus setting the stage for signal initiation. Importantly, both FASN and ErbBs are overexpressed in tumors including OC and represent a new method for treating OC by a combination of inhibitors. A combination of a FASN inhibitor (e.g. C75) and ErbB inhibitor on A2780 ovarian cancer cells (OCC) unexpectedly resulted in inhibited growth of OCC. Interestingly, the combination of C75 and the ErbB inhibitor resulted in a synergistic cell growth inhibition (p<0.01) suggesting cooperation between FASN and ErbB pathways during OCC growth.

### SUMMARY OF THE INVENTION

Accordingly, the invention provides a pharmaceutical composition comprising: a receptor tyrosine kinase inhibitor, or a pharmaceutically-acceptable salt thereof, and a fatty acid synthase inhibitor. In one embodiment, the receptor tyrosine kinase inhibitor is an inhibitor of EGFR, namely an inhibitor of ErbB.

The present invention also provides a pharmaceutical composition comprising: a receptor tyrosine kinase inhibitor, or a pharmaceutically-acceptable salt thereof, a fatty acid synthase inhibitor and a pharmaceutically acceptable carrier.

A method for manufacturing a pharmaceutical composition by combining a receptor tyrosine kinase inhibitor, or a pharmaceutically-acceptable salt thereof, a fatty acid synthase inhibitor and a pharmaceutically acceptable carrier.

A method for treating cancer by administering to a patient a pharmaceutically effective amount of a pharmaceutical composition comprising: a receptor tyrosine kinase inhibitor, or a pharmaceutically-acceptable salt thereof, a fatty acid synthase inhibitor and a pharmaceutically acceptable carrier.

A method for treating ovarian cancer by administering to a patient a pharmaceutically effective amount of a pharmaceutical composition comprising: a receptor tyrosine kinase inhibitor, or a pharmaceutically-acceptable salt thereof, a fatty acid synthase inhibitor and a pharmaceutically acceptable carrier.

In another aspect, the invention provides a method for reducing FASN activity in a cell by contacting the cell with a compound that inhibits ErbB-2 or EGFR activity. In some embodiments, the cell is additionally contacted with a compound that inhibits FASN activity. Activity of FASN, ErbB-2 and EGFR includes (a) downregulation of expression of the polynucleotides encoding FASN, ErbB-2 or EGFR, (b) reduction in the expression of FASN, ErbB-2 or EGFR protein, (c) reduction in the phosphorylation of FASN, ErbB-2 or EGFR, and (d) reduction of downstream signalling of FASN, ErbB-2 or EGFR. Compounds that inhibit FASN, ErbB-2 or EGFR can be biomolecules, such as (a) antibodies or antibody fragments or compositions comprising antibodies that block FASN, ErbB-2 or EGFR signalling, and (b) polynucleotides that inhibit translation activity, such as e.g. siRNAs. In some embodiments, the cell is a cancer cell such as e.g. an ovarian cancer cell or a cervical cancer cell. In some embodiments, the cancer cell is human. In some embodiments, the cancer cell is ex vivo. In other embodiments, the cancer cell is in vivo.

In another aspect, the invention provides a method for reducing ErbB-2 activity in a cell by contacting the cell with a compound that inhibits FASN activity. In some embodiments, the cell is additionally contacted with a compound that inhibits ErbB-2 activity. Activity of FASN and EGFR includes (a) downregulation of expression of the polynucleotides encoding FASN or ErbB-2, (b) reduction in the expression of FASN or ErbB-2 protein, (c) reduction in the phosphorylation of FASN or ErbB-2, and (d) reduction of downstream signalling of FASN or ErbB-2. Compounds that inhibit FASN or ErbB-2 can be biomolecules, such as (a) antibodies or antibody fragments or compositions comprising antibodies that block FASN or ErbB-2 signalling, and (b) polynucleotides that inhibit translation activity, such as e.g. siRNAs. In some embodiments, the cell is a cancer cell such as e.g. an ovarian cancer cell or a cervical cancer cell. In some embodiments, the cancer cell is human. In some embodiments, the cancer cell is ex vivo. In other embodiments, the cancer cell is in vivo.

In another aspect, the invention provides a method for reducing EGFR activity in a cell by contacting the cell with a compound that inhibits FASN activity. In some embodiments, the cell is additionally contacted with a compound that inhibits EGFR activity. Activity of FASN and EGFR includes (a) downregulation of expression of the polynucleotides encoding FASN or EGFR, (b) reduction in the expression of FASN or EGFR protein, (c) reduction in the phosphorylation of FASN or EGFR, and (d) reduction of downstream signalling of FASN or EGFR. Compounds that inhibit FASN or EGFR can be biomolecules, such as (a) antibodies or antibody fragments or compositions comprising antibodies that block FASN or EGFR signalling, and (b) polynucleotides that inhibit translation activity, such as e.g. siRNAs. In some embodiments, the cell is a cancer cell such as e.g. an ovarian cancer cell or a cervical cancer cell. In some embodiments, the cancer cell is human. In some embodiments, the cancer cell is ex vivo. In other embodiments, the cancer cell is in vivo.

In another aspect, the invention provides a method for inhibiting the proliferation of cell(s) by contacting the cell(s) with either (a) a combination a compound that inhibits FASN activity and a compound that inhibits ErbB-2 activity, or (b) a combination a compound that inhibits FASN activity and a compound that inhibits EGFR activity. Activity of FASN, ErbB-2 and EGFR includes (a) downregulation of expression of the polynucleotides encoding FASN, ErbB-2 or EGFR, (b) reduction in the expression of FASN, ErbB-2 or EGFR protein, (c) reduction in the phosphorylation of FASN, ErbB-2 or EGFR, and (d) reduction of downstream signalling of FASN, ErbB-2 or EGFR. Compounds that inhibit FASN, ErbB-2 or EGFR can be biomolecules, such as (a) antibodies or antibody fragments or compositions comprising antibodies that block FASN, ErbB-2 or EGFR signalling, and (b) polynucleotides that inhibit translation activity, such as e.g. siRNAs. In some embodiments, the cell is a cancer cell such as e.g. an ovarian cancer cell or a cervical cancer cell. In some embodiments, the cancer cell is human. In some embodiments, the cancer cell is ex vivo. In other embodiments, the cancer cell is in vivo.

### LISTING OF FIGURES

FIG. 1 depicts a dose-dependent reduction of in-vitro cell growth of A2780 ovarian cancer cells by a combination of a synthetic FASN inhibitor (C75) and of an ErbB inhibitor (EKB-569) as demonstrated by formazan dye assay.

FIG. 2 depicts simultaneous exposure of the cells to the combination of a FASN inhibitor (C75) and the ErbB inhibitor (EKB-569) for 3 days followed by formazan dye assay, indicating that inhibition of FASN and ErbB enzyme cooperatively controls the in-vitro growth of A2780 ovarian cancer cells.

FIG. 3 depicts that inhibition of FASN enzyme activity by C75 down-regulates EGFR gene expression and activity in A2780 ovarian cancer cells.

FIG. 4 depicts that inhibition of FASN enzyme activity by C75 down-regulates ErbB2 protein expression and activity in A2780 ovarian cancer cells.

FIG. 5 depicts that inhibition of FASN enzyme activity by C75 down-regulates FASN expression levels in A2780 ovarian cancer cells.

FIG. 6 depicts that inhibition of EGFR and ErbB2 enzyme activity by EKB-569 down-regulates EGFR expression in A2780 ovarian cancer cells.

FIG. 7 depicts that inhibition of EGFR and ErbB2 enzyme activity by EKB-569 down-regulates ErbB2 expression in A2780 ovarian cancer cells.

FIG. 8 depicts that inhibition of EGFR and ErbB2 enzyme activity by EKB-569 down-regulates FASN expression in A2780 ovarian cancer cells.

FIG. 9 depicts the effects of the synthetic FASN-targeting drug C75, of the ErbB binding small molecule RTKIs pelitinib, canertinib and erlotinib, and of the anti-ErbB antibodies cetuximab, matuzumab and trastuzumab alone or together on in vitro growth of A2780 and SKOV3 ovarian cancer cells as demonstrated by a colorimetric formazan dye assay.

FIG. 10 depicts the down-regulation of FASN mRNA and protein expression by inhibition of FASN activity or by blocking the ErbB system and its downstream pathways as demonstrated by qRT-PCR, branched DNA assay and Western blotting.

FIG. 11 depicts down-regulation of EGFR expression and activity (tyrosine autophosphorylation) by pharmacological inhibition of FASN or ErbB function as demonstrated by RT-PCR followed by agarose gel electrophoresis, qRT-PCR and Western blotting.

FIG. 12 depicts the down-regulation of ErbB2 expression and activity (tyrosyl autophosphorylation) by pharmacological inhibition of FASN or ErbB function as demonstrated by RT-PCR, qRT-PCR and Western blotting.

FIG. 13 depicts the effects of the FASN inhibitor C75 on the activity of the RTK downstream mediators AKT and ERK1/2 in A2780 (left panel) and SKOV3 (right panel) ovarian cancer cell lines as demonstrated by Western blotting and densitometry. Actin was used as loading control and protein bands were related to the corresponding actin bands. Resulting ratios of the vehicle-treated control samples were arbitrarily set at 1.0 and the treated samples were related to the controls (relative levels).

FIG. 14 depicts sequences used in accordance with the invention.

FIG. 15 depicts sequences used in accordance with the invention.

### DETAILED DESCRIPTION OF THE INVENTION

### Definitions

It is to be understood that term “a combination” envisages the simultaneous, sequential or separate administration of the components of the combination. In one aspect of the invention, “a combination” envisages simultaneous administration of the RTK inhibitor and the FASN inhibitor. In a further aspect of the invention, “a combination” envisages sequential administration of the RTK inhibitor and the FASN inhibitor. In another aspect of the invention, “a combination” envisages separate administration of the RTK inhibitor and the FASN inhibitor. The combination of the RTK inhibitor and FASN inhibitor produces a greater effect than that achievable by the administration of either a RTK inhibitor alone or a FASN inhibitor alone. Where the administration of those agents is sequential or separate, the delay in administering the second component should not be such as to lose the benefit of the synergistic effect of the combination therapy. Thus, the present invention provides a combination comprising a receptor tyrosine kinase inhibitor, or a pharmaceutically-acceptable salt thereof, and a FASN inhibitor for use simultaneously, sequentially or separately in the synergistic treatment or prophylaxis of cancer. In one embodiment, the receptor tyrosine kinase inhibitor is an inhibitor of EGFR family, namely an inhibitor of ErbB. In this embodiment, “a combination” envisages simultaneous administration of an EGFR inhibitor (e.g. an ErbB inhibitor) and the FASN inhibitor. The combination also envisages sequential administration of the EGFR inhibitor and the FASN inhibitor. The combination envisages separate administration of the EGFR inhibitor and the FASN inhibitor. The combination of the EGFR inhibitor and FASN inhibitor produces a greater effect than that achievable by the administration of either a EGFR inhibitor alone or a FASN inhibitor alone.

As used herein the term “synergistic combination” refers to the situation where combination of the RTK inhibitor and FASN inhibitor produces a greater effect than the effect achieved by administering the RTK inhibitor alone added to the effect achieved by administering the a FASN inhibitor alone. In one embodiment, the “synergistic combination” refers to the situation where combination of an EGFR inhibitor (e.g. an ErbB inhibitor) and FASN inhibitor produces a greater effect than the effect achieved by administering the EGFR inhibitor alone added to the effect achieved by administering the a FASN inhibitor alone.

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

Suitable examples of FAS inhibitors are described in U.S. Pat. Appl. Publ. No. 20070142456 A1. As used herein, the term “FAS inhibitor” is understood to mean a compound, which directly inhibits the FAS enzyme. Direct inhibition means that the inhibitor reduces FAS activity by direct action on the enzyme rather than as a secondary consequence of some other action of the compound, such as, for example, a reduction in all cellular activities. FAS inhibition can be determined by the means set forth in U.S. Pat. No. 5,981,575.

According to one embodiment, the FAS inhibitor is selected from: tetrahydro-3-methylene-2-oxo-5-n-octyl-4-furancarboxylic acid (C75); cerulenin (2,3-epoxy-4-oxo-7,10-dodecadienoylamide); 1,3-dibromopropanone; Ellman's reagent (5,5′-dithiobis(2-nitrobenzoic acid), DTNB); 4-(4′-chlorobenzyloxy)benzyl nicotinate (KCD-232); 4-(4′-chlorobenzyloxy)benzoic acid (MII); 2(5(4-chlorophenyl)pentyl)oxirane-2-carboxylate (POCA) and its CoA derivative; ethoxyformic anhydride; thiolactomycin; phenyocerulenin; melarsoprol; iodoacetate; phenylarsineoxide; pentostam; melittin; or methyl malonyl CoA. One preferred FAS inhibitor is C75, of formula:

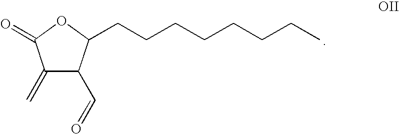

Suitable examples of RTK inhibitors are disclosed in U.S. Pat. Nos. 6,297,258; 6,432,979; 6,617,333; 6,288,082; 6,821,988; 6,521,618; 7,253,286; 7,173,136; 7,105,531; 6,689,772; 6,638,298; 6,596,735; 6,384,051; U.S. Pat. Appl. Publ. Nos. 20080114002; 20080096860; 20080058309; 20080021038; 20070259914; 20070249686; 20070249648; 20070225303; 20070203209; 20070197542; 20070190071; 20070213367; 20070191346; 20070135440; 20070093491; 20070043065; 20070032523; 20060270670; 20060287355; 20060264435; 20060281093; 20060293352; 20060270668; 20060264463; 20060281726; 20060264444; 20060247259; 20060247217; 20060189613; 20060211765; 20060160832; 20060142297; 20060089382; 20060128723; 20060100221; 20060025432; 20060004030; 20050272750; 20050171182; 20050009867; 20040176602; 20040110762; 20040053908; 20030232741; 20030149056; 20020026052; 20010051620 and U.S. patent application Ser. No. 10/939,007.

According to one embodiment, RTK inhibitors are selected from the compounds: 4-Dimethylamino-but-2-enoic acid [4-(3-chloro-4-fluoro-phenylamino)-3-cyano-7-ethoxy-quinolin-6-yl]-amide, (E)-N-{4-[3-chloro-4-(2-pyridinyl methoxy) anilino]-3-cyano-7-ethoxy-6-quinolinyl}-4-(dimethylamino)-2-butenamide, (E)-N-(4-{3-chloro-4-[(3-fluorobenzyl)oxy]anilino}-3-cyano-7-ethoxy-6-quinolinyl)-4-(dimethylamino)-2-butenamide (pelitinib or EKB-569), 4-(2,4-dichloro-5-methoxyanilino)-7-{5-[(4-methyl-1-piperazinyl)methyl]-2-pyridinyl}-3-carbonitrile, canertinib (CI-1033), erlotinib, cetuximab, matuzumab or trastuzumab and pharmaceutically acceptable salts thereof.

Other suitable examples of RTK inhibitors include, but are not limited to, compounds selected from: a) 4-(2,3-dihydro-1H-indol-6-ylamino)-6,7-diethoxy-quinoline-3-carbonitrile or a pharmaceutically acceptable salt thereof; b) 4-(benzothiazol-6-ylamino)-6,7-diethoxy-quinoline-3-carbonitrile or a pharmaceutically acceptable salt thereof; c) 4-(benzo[1,3]dioxol-5-ylamino)-6,7-diethoxy-quinoline-3-carbonitrile or a pharmaceutically acceptable salt thereof; d) 6,7-diethoxy-4-(1H-indazol-6-ylamino)-quinoline-3-carbonitrile or a pharmaceutically acceptable salt thereof; e) 6,7-diethoxy-4-(4-methyl-2-oxo-2H-chromen-7-ylamino)-quinoline-3-carbonitrile or a pharmaceutically acceptable salt thereof; f) 6,7-diethoxy-4-(1H-indol-6-ylamino)-quinoline-3-carbonitrile or a pharmaceutically acceptable salt thereof; g) 6,7-dimethoxy-4-(1H-indazol-6-ylamino)-quinoline-3-carbonitrile or a pharmaceutically acceptable salt thereof; h) 4-(1,3-dioxo-2,3-dihydro-1H-isoindol-5-ylamino)-6,7-diethoxy-quinoline-3-carbonitrile or a pharmaceutically acceptable salt thereof; i) 4-(2,3-dihydro-benzo[1,4]dioxin-6-ylamino)-6,7-diethoxy-quinoline-3-carbonitrile or a pharmaceutically acceptable salt thereof; j) 4-(1H-indazol-6-ylamino)-6,7-bis-(2-methoxy-ethoxy)-quinoline-3-carbonitrile or a pharmaceutically acceptable salt thereof; k) 4-(1,4-dioxo-1,2,3,4-tetrahydro-phthalazin-6-ylamino)-6,7-diethoxy-quinoline-3-c arbonitrile or a pharmaceutically acceptable salt thereof; l) 6,7-diethoxy-4-(indan-5-ylamino)-quinoline-3-carbonitrile or a pharmaceutically acceptable salt thereof; m) 4-(2,4-dioxo-1,4-dihydro-2H-benzo[d][1,3]oxazin-6-ylamino)-6,7-diethoxy-quinoline-3-carbonitrile or a pharmaceutically acceptable salt thereof; n) 6,7-diethoxy-4-(3-oxo-1,3-dihydro-isobenzofuran-5-ylamino)-quinoline-3-carbonitrile or a pharmaceutically acceptable salt thereof; o) 4-(1,1-dioxo-1H-benzo[b]thiophen-6-ylamino)-6,7-diethoxy-quinoline-3-carbonitrile or a pharmaceutically acceptable salt thereof; p) 4-(2,3-dihydro-1H-indol-6-ylamino)-6,7-dimethoxy-quinoline-3-carbonitrile or a pharmaceutically acceptable salt thereof; q) 7-ethoxy-4-(indazol-6-ylamino)-6-methoxy-3-quinolinecarbonitrile or a pharmaceutically acceptable salt thereof; r) 4-(2,3-dihydro-1H-indol-6-ylamino)-6-methoxy-7-(3-pyridin-4-yl-propoxy)-quinoline-3-carbonitrile or a pharmaceutically acceptable salt thereof; s) 9-(1H-indazol-6-ylamino)-2,3-dihydro[1,4]dioxino[2,3-g]quinoline-8-carbonitrile or a pharmaceutically acceptable salt thereof; t) 6,7-diethoxy-4-(1-methyl-2,5-dioxo-2,3,4,5-tetrahydro-1H-benzo[e][1,4]diazepin-7-ylamino)quinoline-3-carbonitrile or a pharmaceutically acceptable salt thereof; u) 4-(1H-indazol-6-ylamino)-6,7,8-trimethoxy-quinoline-3-carbonitrile or a pharmaceutically acceptable salt thereof; v) 6,7-dimethoxy-4-(4-methyl-2-oxo-1,2-dihydro-quinolin-7-ylamino)-quinoline-3-carbonitrile w) 6,7-dimethoxy-4-(2-methyl-benzothiazol-5-ylamino)-quinoline-3-carbonitrile x) 6,7-dimethoxy-4-(2-oxo-2,3-dihydro-benzothiazol-6-ylamino)-quinoline-3-carbonitrile or a pharmaceutically acceptable salt thereof; y) 6,7-dimethoxy-4-(quinolin-5-ylamino)-quinoline-3-carbonitrile or a pharmaceutically acceptable salt thereof; z) 4-(isoquinolin-5-ylamino)-6,7-dimethoxy-quinoline-3-carbonitrile or a pharmaceutically acceptable salt thereof; aa) 6,7-dimethoxy-4-(quinolin-8-ylamino)-quinoline-3-carbonitrile or a pharmaceutically acceptable salt thereof; bb) 4-(8-hydroxyquinolin-5-ylamino)-6,7-dimethoxy-quinoline-3-carbonitrile or a pharmaceutically acceptable salt thereof; cc) 4-(1H-indol-4-ylamino)-6,7-dimethoxy-quinoline-3-carbonitrile or a pharmaceutically acceptable salt thereof; dd) 4-(1H-indazol-5-ylamino)-6,7-dimethoxy-quinoline-3-carbonitrile or a pharmaceutically acceptable salt thereof; ee) 4-(1H-indazol-6-ylamino)-5,8-dimethoxy-quinoline-3-carbonitrile or a pharmaceutically acceptable salt thereof; ff) 4-(1H-indazol-6-ylamino)-7-methoxy-6-(3-morpholin-4-yl-propoxy)-quinoline-3-carbonitrile or a pharmaceutically acceptable salt thereof; gg) 4-(3H-benzotriazol-5-ylamino)-7-methoxy-6-(3-morpholin-4-yl-propoxy)quinoline-3-carbonitrile or a pharmaceutically acceptable salt thereof; hh) 4-(1H-indazol 6-ylamino)-6-methoxy-quinoline-3-carbonitrile or a pharmaceutically acceptable salt thereof; ii) 4-(3H-benzotriazol-5-ylamino)-6-methoxy-quinoline-3-carbonitrile or a pharmaceutically acceptable salt thereof; jj) 4-(1H-indazol-6-ylamino)-7-methoxy-quinoline-3-carbonitrile or a pharmaceutically acceptable salt thereof; kk) 4-(3H-benzotriazol-5-ylamino)-7-methoxyquinoline-3-carbinitile or a pharmaceutically acceptable salt thereof; ll) 7-hydroxy-4-(1H-indazol-6-ylamino)-quinoline-3-carbonitrile or a pharmaceutically acceptable salt thereof; mm) 4-(1H-indol-5-ylamino)-7-methoxy-quinoline-3-carbonitrile or a pharmaceutically acceptable salt thereof; nn) 7-hydroxy-4-(3H-benzotrizol-5-ylamino)-quinoline-3-carbonitrile or a pharmaceutically acceptable salt thereof; oo) 4-(1H-indazol-6-ylamino)-8-methoxy-quinoline-3-carbonitrile or a pharmaceutically acceptable salt thereof; pp) 4-(3H-benzotriazol-5-ylamino)-8-methoxy-quinoline-3-carbonitrile or a pharmaceutically acceptable salt thereof; qq) 4-(1H-indol-5-ylamino)-6,7-Dimethoxy-quinoline-3-carbonitrile or a pharmaceutically acceptable salt thereof; rr) 4-(1H-benzoimidazol-5-ylamino)-6,7-dimethoxy-quinoline-3-carbonitrile or a pharmaceutically acceptable salt thereof; ss) 6,7-dimethoxy-4-(2-methyl-1H-benzoimidazol-5-ylamino)-quinoline-3-carbonitrile or a pharmaceutically acceptable salt thereof; tt) 6,7-dimethoxy-4-(quinoline-6-ylamino)-quinoline-3-carbonitrile or a pharmaceutically acceptable salt thereof; uu) 4-(4-chloronaphthalen-1-ylamino)-6,7-Dimethoxy-quinoline-3-carbonitrile or a pharmaceutically acceptable salt thereof; vv) 6,7-dimethoxy-4-(5,6,7,8,-tetrahydro-naphthalen-1-ylamino)-quinoline-3-carbonitrile or a pharmaceutically acceptable salt thereof; ww) 4-(3H-benzotriazol-5-ylamino)-6,7,8-Trimethoxy-quinoline-3-carbonitrile or a pharmaceutically acceptable salt thereof; xx) 4-(1H-indazol-6-ylamino)-6-methoxy-7-[2-(4-methyl-piperazin-1-yl)-ethoxy]-quinoline-3-carbonitrile or a pharmaceutically acceptable salt thereof; yy) 7-{2-[(2-hydroxy-ethyl)-amino]-ethoxy}-4(1H-indazol-6-ylamino)-6-methoxy-quinoline-3-carbonitrile or a pharmaceutically acceptable salt thereof; zz) 7-{2-[bis-(2-hydroxy-ethyl)-amino]-ethoxy}-4-(1H-indazol-6-ylamino)-6-methoxy-quinoline-3-carbonitrile or a pharmaceutically acceptable salt thereof; aaa) 7-[2-(4-hydroxypiperidin-1-yl)-ethoxy]-4-(1H-indazol-6-ylamino)-6-methoxy-quinoline-3-carbonitrile or a pharmaceutically acceptable salt thereof; bbb) 7-{2-[(4-(2-hydroxy-ethyl)-piperazin-1-yl)-ethoxy]-4-(1H-indazol-6-ylamino)-6-methoxy-quinoline-3-carbonitrile or a pharmaceutically acceptable salt thereof; ccc) 7-[2-(1,4dioxa-8-aza-spiro[4,5]dec-8-yl)-ethoxy]-4-(1H-indazol-6-ylamino)-6-methoxy-quinoline-3-carbonitrile or a pharmaceutically acceptable salt thereof; ddd) 7-[2-([1,3]dioxolan-2-ylmethyl-methyl-amino)-ethoxy]-4-(1H-indazol-6-ylamino)-6-methoxy-quinoline-3-carbonitrile eee) 7-[2-(3,4-dihydro-1H-isoquinolin-2-yl)-ethoxy]-4-(1H-indazol-6-ylamino)-6-methoxy-quinoline-3-carbonitrile or a pharmaceutically acceptable salt thereof; fff) 4-(1H-indazol-6-ylamino)-6-methoxy-7-(2-thiomorpholin-4-yl-ethoxy)-quinoline-3-carbonitrile or a pharmaceutically acceptable salt thereof; ggg) 7-(2-chloroethoxy)-4-(1H-indazol-6-ylamino)-6-methoxyquinoline-3-carbonitrile or a pharmaceutically acceptable salt thereof; hhh) 7-(2-dimethylaminoethoxy)-4-(1H-indazol-6-ylamino)-6-methoxyquinoline-3-carbonitrile or a pharmaceutically acceptable salt thereof; iii) 4-(1H-indazol-6-ylamino)-6-methoxy-7-(2-morpholin-4-yl-ethoxy)quinoline-3-carbonitrile or a pharmaceutically acceptable salt thereof; jjj) 4-(3H-benzotriazol-5-ylamino)-7-(2-chloroethoxy)-6-methoxyquinoline-3-carbonitrile or a pharmaceutically acceptable salt thereof; kkk) 7-(3-chloropropoxy)-4-(1H-indazol-6-ylamino)-6-methoxyquinoline-3-carbonitrile or a pharmaceutically acceptable salt thereof; lll) 4-(1H-indazole-6-ylamino)-6-methoxy-7-(3-morpholin-4-ylpropoxy)-quinoline-3-carbonitrile or a pharmaceutically acceptable salt thereof; mmm) 4-[3-chloro-4-(1-methyl-2-imidazolylthio) phenylamino]-6,7-diethoxy-3-quinolinecarbonitrile or a pharmaceutically acceptable salt thereof; nnn) 4-[3-chloro-4-(1-methyl-2-imidazolylthio) phenylamino]-6,7-dimethoxy-3-quinolinecarbonitrile or a pharmaceutically acceptable salt thereof; ooo) 6-amino-4-[3-chloro-4-(1-methyl-1H-imidazol-2-ylsulfanyl)-phenylamino]-quinoline-3-carbonitrile or a pharmaceutically acceptable salt thereof; ppp) N-{4-[3-chloro-4-(1-methyl-1H-imidazol-2-ylsulfanyl)-phenylamino]-3-cyano-quinolin-6-yl}-acrylamide or a pharmaceutically acceptable salt thereof; qqq) 6-amino-4-(1H-indol-5-ylamino)-quinoline-3-carbonitrile or a pharmaceutically acceptable salt thereof; rrr) 4-(1H-indol-5-ylamino)-6-nitro-quinoline-3-carbonitrile or a pharmaceutically acceptable salt thereof; sss) 4-(2-Hydroxy-naphthalen-1-ylamino)-6,7-dimethoxy-quinoline-3-carbonitrile or a pharmaceutically acceptable salt thereof; ttt) 4-(2,3-Dihydro-benzo[1,4]dioxin-6-ylamino)-6,7-dimethoxy-quinoline-3-carbonitrile or a pharmaceutically acceptable salt thereof; uuu) 4-(2-Mercapto-benzothiazol-6-ylamino)-6,7-dimethoxy-quinoline-3-carbonitrile or a pharmaceutically acceptable salt thereof; vvv) 4-(6-Hydroxy-naphthalen-1-ylamino)-6,7-dimethoxy-quinoline-3-carbonitrile or a pharmaceutically acceptable salt thereof; www) 4-(1H-Indazol-6-ylamino)-5-methoxy-quinoline-3-carbonitrile or a pharmaceutically acceptable salt thereof; xxx) 4-(2-chloro-5-methoxyanilino)-5-methoxyquinoline-3-carbonitrile or a pharmaceutically acceptable salt thereof; yyy) 4-[(2-Amino-4-chlorophenyl)amino]-6,7-dimethoxy-3-quinolinecarbonitrile or a pharmaceutically acceptable salt thereof; zzz) 4-[(3-hydroxy-2-naphthyl)amino]-6,7-dimethoxy-3-quinolinecarbonitrile or a pharmaceutically acceptable salt thereof; aaaa) 4-{3-chloro-4-[(1-methyl-1H-imidazol-2-yl)sulfanyl]anilino}-7-methoxy-6-nitro-3-quinolinecarbonitrile or a pharmaceutically acceptable salt thereof; bbbb) 6-amino-4-{3-chloro-4-[(1-methyl-1H-imidazol-2-yl)sulfanyl]anilino}-7-methoxy-3-quinolinecarbonitrile or a pharmaceutically acceptable salt thereof; cccc) (E)-N-(4-{3-chloro-4-[(1-methyl-1H-imidazol-2-yl) sulfanyl]anilino}-3-cyano-7-methoxy-6-quinolinyl)-4-(dimethylamino)-2-butenamide or a pharmaceutically acceptable salt thereof; dddd) 4-[3-chloro-4-(1,3-thiazol-2-ylsulfanyl)anilino]-7-methoxy-6-nitro-3-quinolinecarbonitrile or a pharmaceutically acceptable salt thereof; eeee) 6-amino-4-[3-chloro-4-(1,3-thiazol-2-ylsulfanyl) anilino]-7-methoxy-3-quinolinecarbonitrile or a pharmaceutically acceptable salt thereof; ffff) (E)-N-{4-[3-chloro-4-(1,3-thiazol-2-ylsulfanyl) anilino]-3-cyano-7-methoxy-6-quinolinyl}-4-(dimethylamino)-2-butenamide or a pharmaceutically acceptable salt thereof; gggg) 4-[3-chloro-4-(1H-imidazol-1-yl)anilino]-7-methoxy-6-nitro-3-quinolinecarbonitrile or a pharmaceutically acceptable salt thereof; hhhh) 6-amino-4-[3-chloro-4-(1H-imidazol-1-yl)anilino]-7-methoxy-3-quinolinecarbonitrile or a pharmaceutically acceptable salt thereof; iiii) (E)-N-{4-[3-chloro-4-(1H-imidazol-1-yl)anilino]-3-cyano-7-methoxy-6-quinolinyl}-4-(dimethylamino)-2-butenamide or a pharmaceutically acceptable salt thereof; jjjj) 4-{3-chloro-4-[(4-oxo-3,4-dihydro-2-quinazolinyl) sulfanyl]anilino}-7-methoxy-6-nitro-3-quinolinecarbonitrile or a pharmaceutically acceptable salt thereof; kkkk) 6-amino-4-3-chloro-4-[(4-oxo-3,4-dihydro-2-quinazolinyl)sulfanyl]anilino)-7-methoxy-3-quinolinecarbonitrile or a pharmaceutically acceptable salt thereof; llll) (E)-N-(4-{3-chloro-4-[(4-oxo-3,4-dihydro-2-quinazolinyl)sulfanyl]anilino}-3-cyano-7-methoxy-6-quinolinyl)-4-(dimethylamino)-2-butenamide or a pharmaceutically acceptable salt thereof; mmmm) 6-methoxy-7-[3-(4-morpholinyl)propoxy]-4-[4-(4-pyridinylmethyl)anilino]-3-quinolinecarbonitrile or a pharmaceutically acceptable salt thereof; nnnn) 6-methoxy-7-[3-(4-morpholinyl)propoxy]-4-[4-(3-pyridinylmethyl)anilino]-3-quinolinecarbonitrile or a pharmaceutically acceptable salt thereof; oooo) 6-methoxy-7-[3-(4-morpholinyl)propoxy]-4-[4-(2-pyridinylmethyl)anilino]-3-quinolinecarbonitrile or a pharmaceutically acceptable salt thereof; pppp) (E)-N-(4-{4-[acetyl(3-pyridinylmethyl)amino]-3-chloroanilino}-3-cyano-7-methoxy-6-quinolinyl)-4-(dimethylamino)-2-butenamide or a pharmaceutically acceptable salt thereof; qqqq) N-(2-chloro-4-[(3-cyano-7-methoxy-6-nitro-4-quinolinyl)amino]phenyl}-N-(3-pyridinylmethyl)acetamide or a pharmaceutically acceptable salt thereof; rrrr) N-{4-[(6-amino-3-cyano-7-methoxy-4-quinolinyl)amino]-2-chlorophenyl}-N-(3-pyridinylmethyl)acetamide or a pharmaceutically acceptable salt thereof; ssss) N-(4-{[6-(acetylamino)-3-cyano-7-methoxy-4-quinolinyl]amino}-2-chlorophenyl)-N-(3-pyridinylmethyl)acetamide or a pharmaceutically acceptable salt thereof; tttt) 4-[3-chloro-4-(1,3-dimethyl-2,4,6-trioxohexahydro-5-pyrimidinyl)anilino]-7-methoxy-6-nitro-3-quinolinecarbonitrile or a pharmaceutically acceptable salt thereof; uuuu) 4-{3-chloro-4-[(4-phenyl-1,3-thiazol-2-yl)sulfanyl]anilino}-7-methoxy-6-nitro-3-quinolinecarbonitrile or a pharmaceutically acceptable salt thereof; vvvv) 6-methoxy-7-[3-(4-morpholinyl)propoxy]-4-[4-(3-thienylmethyl)anilino]-3-quinolinecarbonitrile or a pharmaceutically acceptable salt thereof; wwww) 6-methoxy-7-[3-(4-morpholinyl)propoxy]-4-[4-(2-thienylmethyl)anilino]-3-quinolinecarbonitrile or a pharmaceutically acceptable salt thereof; xxxx) 6-methoxy-4-(4-phenoxyanilino)-7-[2-(2H-1,2,3-triazol-2-yl)ethoxy]-3-quinolinecarbonitrile or a pharmaceutically acceptable salt thereof; yyyy) 6-methoxy-4-(4-phenoxyanilino)-7-[2-(1H-1,2,3-triazol-1-yl)ethoxy]-3-quinolinecarbonitrile or a pharmaceutically acceptable salt thereof; zzzz) 4-(4-benzylanilino)-6-methoxy-7-[2-(2H-1,2,3-triazol-2-yl)ethoxy]-3-quinolinecarbonitrile or a pharmaceutically acceptable salt thereof; aaaaa) 4-(4-benzylanilino)-6-methoxy-7-[2-(H-1,2,3-triazol-1-yl)ethoxy]-3-quinolinecarbonitrile or a pharmaceutically acceptable salt thereof; bbbbb) 6-methoxy-7-[3-(4-morpholinyl)propoxy]-4-[4-(2-pyridinyloxy)anilino]-3-quinoline carbonitrile or a pharmaceutically acceptable salt thereof; ccccc) 4-{3-chloro-4-[(1-methyl-1H-imidazol-2-yl)sulfanyl]anilino}-6-methoxy-7-[3-(4-morpholinyl)propoxy]-3-quinolinecarbonitrile or a pharmaceutically acceptable salt thereof; ddddd) 4-[4-(2-furylmethyl)anilino]-6-methoxy-7-[3-(4-morpholinyl)propoxy]-3-quinolinecarbonitrile or a pharmaceutically acceptable salt thereof; eeeee) 6-methoxy-7-[3-(4-morpholinyl)propoxy]-4-[4-(tetrahydro-2-furanylmethyl)anilino]-3-quinolinecarbonitrile or a pharmaceutically acceptable salt thereof; fffff) 4-[4-(3-furylmethyl)anilino]-6-methoxy-7-[3-(4-morpholinyl)propoxy]-3-quinolinecarbonitrile or a pharmaceutically acceptable salt thereof; ggggg) 6-methoxy-7-[3-(4-morpholinyl)propoxy]-4-[4-(tetrahydro-3-furanylmethyl)anilino]-3-quinolinecarbonitrile or a pharmaceutically acceptable salt thereof; hhhhh) 4-(3-chloro-4-{[5-(trifluoromethyl)-1,3,4-thiadiazol-2-yl]amino}anilino)-7-ethoxy-6-nitro-3-quinolinecarbonitrile or a pharmaceutically acceptable salt thereof; iiiii) (E)-N-[4-(3-chloro-4-{[5-(trifluoromethyl)-1,3,4-thiadiazol-2-yl]amino}anilino)-3-cyano-7-ethoxy-6-quinolinyl]-4-(dimethylamino)-2-butenamide or a pharmaceutically acceptable salt thereof; jjjjj) 4-[3-chloro-4-(4-pyridinyloxy)anilino]-7-ethoxy-6-nitro-3-quinolinecarbonitrile or a pharmaceutically acceptable salt thereof; kkkkk) 6-amino-4-[3-chloro-4-(4-pyridinyloxy)anilino]-7-ethoxy-3-quinolinecarbonitrile or a pharmaceutically acceptable salt thereof; lllll) (E)-N-{4-[3-chloro-4-(4-pyridinyloxy)anilino]-3-cyano-7-ethoxy-6-quinolinyl}-4-(dimethylamino)-2-butenamide or a pharmaceutically acceptable salt thereof; mmmmm) 4-{3-chloro-4-[(3-pyridinylmethyl)amino]anilino}-7-methoxy-6-nitro-3-quinolinecarbonitrile or a pharmaceutically acceptable salt thereof; nnnnn) 6-amino-4-{3-chloro-4-[(4-phenyl-1,3-thiazol-2-yl)sulfanyl]anilino}-7-methoxy-3-quinolinecarbonitrile or a pharmaceutically acceptable salt thereof; ooooo) 6-amino-4-(3-chloro-4-{[5-(trifluoromethyl)-1,3,4-thiadiazol-2-yl]amino}anilino)-7-ethoxy-3-quinolinecarbonitrile or a pharmaceutically acceptable salt thereof; ppppp) 6-methoxy-7-[3-(4-morpholinyl)propoxy]-4-[4-(2-phenylethyl)anilino]-3-quinolinecarbonitrile or a pharmaceutically acceptable salt thereof; qqqqq) (E)-N-(4-{3-chloro-4-[(4-phenyl-1,3-thiazol-2-yl) sulfanyl]anilino}-3-cyano-7-methoxy-6-quinolinyl)-4-(dimethylamino)-2-butenamide or a pharmaceutically acceptable salt thereof; rrrrr) 4-[3-chloro-4-(1H-imidazol-1-yl)anilino]-6-methoxy-7-[3-(4-morpholinyl)propoxy]-3-quinolinecarbonitrile or a pharmaceutically acceptable salt thereof; sssss) 6-methoxy-7-[3-(4-morpholinyl)propoxy]-4-[4-(3-pyridinyloxy)anilino]-3-quinoline carbonitrile or a pharmaceutically acceptable salt thereof; ttttt) 4-[3-chloro-4-(4-pyridinyloxy)anilino]-6-methoxy-7-[3-(4-morpholinyl)propoxy]-3-quinolinecarbonitrile or a pharmaceutically acceptable salt thereof; uuuuu) 6-methoxy-7-[3-(4-morpholinyl)propoxy]-4-[4-(4-pyridinyloxy)anilino]-3-quinoline carbonitrile or a pharmaceutically acceptable salt thereof; vvvvv) 4-[2-chloro-4-(1,3-thiazol-2-ylsulfanyl)anilino]-6-methoxy-7-[3-(4-morpholinyl)propoxy]-3-quinolinecarbonitrile or a pharmaceutically acceptable salt thereof; wwwww) N-[2-chloro-4-({3-cyano-6-methoxy-7-[3-(4-morpholinyl)propoxy]-4-quinolinyl}amino)phenyl]-N-(3-pyridinylmethyl)acetamide or a pharmaceutically acceptable salt thereof; xxxxx) 6-methoxy-7-[3-(4-morpholinyl)propoxy]-4-[4-(1H-tetraazol-5-ylmethyl)anilino]-3-quinolinecarbonitrile or a pharmaceutically acceptable salt thereof; yyyyy) 6-methoxy-7-[3-(4-morpholinyl)propoxy]-4-[4-(2H-1,2,3-triazol-2-ylmethyl)anilino]-3-quinolinecarbonitrile or a pharmaceutically acceptable salt thereof; zzzzz) 6-methoxy-7-[3-(4-morpholinyl)propoxy]-4-[4-(1H-1,2,3-triazol-1-ylmethyl)anilino]-3-quinolinecarbonitrile or a pharmaceutically acceptable salt thereof; aaaaaa) 4-(2,4-dichloro-5-methoxyanilino)-6-methoxy-7-[2-(2H-1,2,3-triazol-2-yl)ethoxy]-3-quinolinecarbonitrile or a pharmaceutically acceptable salt thereof; bbbbbb) 4-(2,4-dichloro-5-methoxyanilino)-6-methoxy-7-[2-(1H-1,2,3-triazol-1-yl)ethoxy]-3-quinolinecarbonitrile or a pharmaceutically acceptable salt thereof; cccccc) 7-ethoxy-6-nitro-4-[4-[(4-phenyl-1,3-thiazol-2-yl)sulfanyl]-3-(trifluoromethyl)anilino]-3-quinolinecarbonitrile or a pharmaceutically acceptable salt thereof; dddddd) 6-amino-7-ethoxy-4-[4-[(4-phenyl-1,3-thiazol-2-yl)sulfanyl]-3-(trifluoromethyl)anilino]-3-quinolinecarbonitrile or a pharmaceutically acceptable salt thereof; eeeeee) (E)-N-{3-cyano-7-ethoxy-4-[4-[(4-phenyl-1,3-thiazol-2-yl)sulfanyl]-3-(trifluoromethyl)anilino]-6-quinolinyl}-4-(dimethylamino)-2-butenamide or a pharmaceutically acceptable salt thereof; ffffff) 4-[3-chloro-4-(1H-imidazol-1-ylmethyl)anilino]-7-ethoxy-6-nitro-3-quinolinecarbonitrile or a pharmaceutically acceptable salt thereof; gggggg) 6-amino-4-[3-chloro-4-(1H-imidazol-1-ylmethyl)anilino]-7-ethoxy-3-quinolinecarbonitrile or a pharmaceutically acceptable salt thereof; hhhhhh) (E)-N-{4-[3-chloro-4-(1H-imidazol-1-ylmethyl)anilino]-3-cyano-7-ethoxy-6-quinolinyl}-4-(dimethylamino)-2-butenamide or a pharmaceutically acceptable salt thereof; iiiiii) 4-{3-chloro-4-[(4-methyl-2-pyrimidinyl)sulfanyl]anilino}-7-ethoxy-6-nitro-3-quinolinecarbonitrile or a pharmaceutically acceptable salt thereof; jjjjjj) 6-amino-4-{3-chloro-4-[(4-methyl-2-pyrimidinyl)sulfanyl]anilino}-7-ethoxy-3-quinolinecarbonitrile or a pharmaceutically acceptable salt thereof; kkkkkk) (E)-N-(4-{3-chloro-4-[(4-methyl-2-pyrimidinyl)sulfanyl]anilino}-3-cyano-7-ethoxy-6-quinolinyl)-4-(dimethylamino)-2-butenamide or a pharmaceutically acceptable salt thereof; llllll) 4-{3-chloro-4-[(4,6-dimethyl-2-pyrimidinyl)sulfanyl]anilino}-7-ethoxy-6-nitro-3-quinolinecarbonitrile or a pharmaceutically acceptable salt thereof; mmmmmm) 6-amino-4-{3-chloro-4-[(4,6-dimethyl-2-pyrimidinyl)sufanyl]anilino}-7-ethoxy-3-quinolinecarbonitrile or a pharmaceutically acceptable salt thereof; nnnnnn) (E)-N-(4-{3-chloro-4-[(4,6-dimethyl-2-pyrimidinyl)sulfanyl]anilino}-3-cyano-7-ethoxy-6-quinolinyl)-4-(dimethylamino)-2-butenamide or a pharmaceutically acceptable salt thereof; oooooo) 4-[4-(1H-imidazol-2-ylmethyl)anilino]-6-methoxy-7-[3-(4-morpholinyl)propoxy]-3-quinolinecarbonitrile or a pharmaceutically acceptable salt thereof; pppppp) 6-methoxy-7-[3-(4-morpholinyl)propoxy]-4-[4-(1H-tetraazol-1-ylmethyl)anilino]-3-quinolinecarbonitrile or a pharmaceutically acceptable salt thereof; qqqqqq) 6-methoxy-7-[3-(4-morpholinyl)propoxy]-4-[4-(2H-tetraazol-2-ylmethyl)anilino]-3-quinolinecarbonitrile or a pharmaceutically acceptable salt thereof; rrrrrr) 4-{3-chloro-4-[(4,6-dimethyl-2-pyrimidinyl)sulfanyl]anilino}-6-methoxy-7-[3-(4-morpholinyl)propoxy]-3-quinolinecarbonitrile or a pharmaceutically acceptable salt thereof; ssssss) 4-{3-chloro-4-[(4-methyl-2-pyrimidinyl)sulfanyl]anilino}-6-methoxy-7-[3-(4-morpholinyl)propoxy]-3-quinolinecarbonitrile or a pharmaceutically acceptable salt thereof; tttttt) (E)-N-[4-(3-chloro-4-{[2-(phenylsulfanyl)acetyl}amino]anilino)-3-cyano-7-methoxy-6-quinolinyl]-4-(dimethylamino)-2-butenamide or a pharmaceutically acceptable salt thereof; uuuuuu) 4-[4-(2,6-dimethoxyphenoxy)anilino]-6-methoxy-7-[3-(4-morpholinyl)propoxy]-3-quinolinecarbonitrile or a pharmaceutically acceptable salt thereof; vvvvvv) 6-methoxy-4-[4-(3-methoxyphenoxy)anilino]-7-[3-(4-morpholinyl)propoxy]-3-quinolinecarbonitrile or a pharmaceutically acceptable salt thereof; wwwwww) 6-methoxy-4-{4-[(1-methyl-1H-imidazol-2-yl)sulfanyl]anilino}-7-[3-(4-morpholinyl)propoxy]-3-quinolinecarbonitrile or a pharmaceutically acceptable salt thereof; xxxxxx) (E)-N-{4-[3-chloro-4-(1,3-thiazol-2-ylsulfanyl)anilino]-3-cyano-7-methoxy-6-quinolinyl}-4-[(2-methoxyethyl)(methyl)amino]-2-but enamide or a pharmaceutically acceptable salt thereof; yyyyyy) (E)-N-(4-{3-chloro-4-[(5-phenyl-1,3-thiazol-2-yl) sulfanyl]anilino}-3-cyano-7-methoxy-6-quinolinyl)-4-(dimethylamino)-2-butenamide or a pharmaceutically acceptable salt thereof; zzzzzz) (E)-N-(4-{3-chloro-4-[(4-phenyl-1,3-thiazol-2-yl) sulfanyl]anilino}-3-cyano-7-‘ethoxy-6-quinolinyl)-4-(dimethylamino)-2-butenamide or a pharmaceutically acceptable salt thereof; aaaaaaa) 4-{3-chloro-4-[(4,6-dimethyl-2-pyrimidinyl)sulfanyl]anilino}-6,7-dimethoxy-3-quinolinecarbonitrile or a pharmaceutically acceptable salt thereof; bbbbbbb) 6,7-dimethoxy-4-({6-[(4-phenyl-1,3-thiazol-2-yl)sulfanyl]-3-pyridinyl}amino)-3-quinolinecarbonitrile or a pharmaceutically acceptable salt thereof; or ccccccc) 4-{3-chloro-4-[(1-methyl-1H-imidazol-2-yl) sulfanyl]anilino}-6-methoxy-7-[3-(1H-1,2,3-triazol-1-yl)propoxy]-3-quinolinecarbonitrile or a pharmaceutically acceptable salt thereof.

Other suitable examples of RTK inhibitors include, but are not limited to compounds selected from: a) 1-Methyl-1,2,5,6-tetrahydro-pyridine-3-carboxylic acid [4-(3-bromo-phenylamino)-3-cyano-quinolin-6-yl]-amide or a pharmaceutically acceptable salt thereof; b) N-[4-[(3-Bromophenyl)amino]-3-cyano-6-quinolinyl]-4-(N-allyl-N-methylamino)-2-butynamide or a pharmaceutically acceptable salt thereof; c) N-[4-[(3-Bromophenyl)amino]-3-cyano-6-quinolinyl]-4-(N-methoxyethyl-N-methylamino)-2-butynamide or a pharmaceutically acceptable salt thereof; d) N-[4-[(3-Bromophenyl)amino]-3-cyano-6-quinolinyl]-4-(bis-(2-methoxyethyl)amino)-2-butynamide or a pharmaceutically acceptable salt thereof; e) 4-Methoxymethoxy-but-2-ynoic acid[4-(3-bromo-phenylamino)-3-cyano-quinolin-6-yl]-amide or a pharmaceutically acceptable salt thereof; f) 4-(4-Chloro-2-fluoro-phenylamino)-6-methoxy-7-(2-pyridin-4-yl-ethoxy)-1-quinoline-3-carbonitrile or a pharmaceutically acceptable salt thereof; g) 4-(2-Methoxy-ethoxy)-but-2-ynoic acid[4-(3-bromo-phenylamino)-3-cyano-quinolin-6-yl]-amide or a pharmaceutically acceptable salt thereof; h) 4-((2S)-2-Methoxymethylpyrrolidin-1-yl)but-2-ynoic Acid[4-(3-bromophenylamino)-3-cyanoquinolin-6-yl]amide or a pharmaceutically acceptable salt thereof; i) 4-(1,4-Dioxa-8-azaspiro[4,5]dec-8-yl)but-2-ynoic Acid[4-(3-Bromophenylamino)-3-cyanoquinolin-6-yl]amide or a pharmaceutically acceptable salt thereof; j) 4-(3-Bromo-phenylamino)-6-(2-ethoxy-3,4-dioxo-cyclobut-1-enylamino)-quinoline-3-carbonitrile or a pharmaceutically acceptable salt thereof; k) 4-[(2-Methoxy-ethyl)-methyl-amino]-but-2-enoic acid[4-(3-chloro-4-fluoro-phenylamino)-3-cyano-7-methoxy-quinolin-6-yl]-amide or a pharmaceutically acceptable salt thereof; l) (S)-4-(2-Methoxymethyl-pyrrolidin-1-yl)-but-2-enoic acid[4-(3-chloro-4-fluorophenylamino)-3-cyano-7-methoxy-quinolin-6-yl]-amide dihydrochloride or a pharmaceutically acceptable salt thereof; m) 4-(3-Hydroxymethyl-piperidin-1-yl)-but-2-enoic acid[4-(3-chloro-4-fluoro-phenylamino)-3-cyano-7-methoxy-quinolin-6-yl]-amide or a pharmaceutically acceptable salt thereof; n) 4-(1,4-Dioxa-8-aza-spiro[4.5]dec-8-yl)-but-2-enoic acid[4-(3-chloro-4-fluorophenylamino)-3-cyano-7-methoxy-quinolin-6-yl]-amide or a pharmaceutically acceptable salt thereof; o) 4-(2-Hydroxymethyl-piperidin-1-yl)-but-2-enoic acid[4-(3-chloro-4-fluorophenylamino)-3-cyano-7-methoxy-quinolin-6-yl]-amide or a pharmaceutically acceptable salt thereof; p) 4-Bromo-but-2-enoic acid[4-(3-chloro-4-fluoro-phenylamino)-3-cyano-7-methoxyquinolin-6-yl]-amide or a pharmaceutically acceptable salt thereof; q) 4-(3-hydroxy-4-methyl-phenylamino)-6-methoxy-7-(3-pyridin-4-yl-propoxy)-quinoline-3-carbonitrile or a pharmaceutically acceptable salt thereof; r) 4-Diallylamino-but-2-enoic acid[4-(3-chloro-4-fluoro-phenylamino)-3-cyano-7-methoxy-quinolin-6-yl]-amide or a pharmaceutically acceptable salt thereof; s) 4-[Bis-(2-methoxy-ethyl)-amino]-but-2-enoic acid[4-(3-chloro-4-fluoro-phenylamino)-3-cyano-7-methoxy-quinolin-6-yl]-amide or a pharmaceutically acceptable salt thereof; t) 4-([1,3]Dioxolan-2-ylmethyl-methyl-amino)-but-2-enoic acid3-cyano-7-methoxy-quinolin-6-yl]-amide or a pharmaceutically acceptable salt thereof; u) 4-[Bis-(2-hydroxy-ethyl)-amino]-but-2-enoic acid [4-(3-chloro-4-fluoro-phenylamino)-3-cyano-7-methoxy-quinolin-6-yl]-amide or a pharmaceutically acceptable salt thereof; v) 4-Thiomorpholin-4-yl-but-2-enoic acid[4-(3-chloro-4-fluoro-phenylamino)-3-cyano-7-methoxy-quinolin-6-yl]-amide or a pharmaceutically acceptable salt thereof; w) 4-[4-(2-Hydroxy-ethyl)-piperazin-1-yl]-but-2-enoic acid[4-(3-chloro-4-fluoro-phenylamino)-3-cyano-7-methoxy-quinolin-6-yl]-amide or a pharmaceutically acceptable salt thereof; x) 4-(1,4,7-Trioxa-10-aza-cyclododec-10-yl)-but-2-enoic acid[4-(3-chloro-4-fluoro-phenylamino)-3-cyano-7-methoxy-quinolin-6-yl]-amide or a pharmaceutically acceptable salt thereof; y) 4-(Methoxy-methyl-amino)-but-2-enoic acid[4-(3-chloro-4-fluoro-phenylamino)-3-cyano-7-methoxy-quinolin-6-yl]-amide or a pharmaceutically acceptable salt thereof; z) 4-(4-Hydroxy-piperidin-1-yl)-but-2-enoic acid [4-(3-chloro-4-fluoro-phenylamino)-3-cyano-7-methoxy-quinolin-6-yl]-amide or a pharmaceutically acceptable salt thereof; aa) 4-[1,4]Bipiperidinyl-1′-yl-but-2-enoic acid[4-(3-chloro-4-fluoro-phenylamino)-3-cyano-7-methoxy-quinolin-6-yl]-amide or a pharmaceutically acceptable salt thereof; bb) 4-Thiazolidin-3-yl-but-2-enoic acid[4-(3-chloro-4-fluoro-phenylamino)-3-cyano-7-methoxy-quinolin-6-yl]-amide or a pharmaceutically acceptable salt thereof; cc) 3-{3-[4-(3-Chloro-4-fluoro-phenylamino)-3-cyano-7-methoxy-quinolin-6-ylcarbamoyl]-allyl}-4-methyl-thiazol-3-ium bromide or a pharmaceutically acceptable salt thereof; dd) 4-(2,6-Dimethyl-piperidin-1-yl)-but-2-enoic acid[4-(3-chloro-4-fluoro-phenylamino)-3-cyano-7-methoxy-quinolin-6-yl]-amide or a pharmaceutically acceptable salt thereof; ee) 4-[Bis-(2-hydroxy-propyl)-amino]-but-2-enoic acid [4-(3-chloro-4-fluoro-phenylamino)-3-cyano-7-methoxy-quinolin-6-yl]-amide or a pharmaceutically acceptable salt thereof; ff) 4-(3-Hydroxy-pyrrolidin-1-yl)-but-2-enoic acid[4-(3-chloro-4-fluoro-phenylamino)-3-cyano-7-methoxy-quinolin-6-yl]-amide or a pharmaceutically acceptable salt thereof; gg) 4-[(2-Hydroxy-ethyl)-methyl-amino]-but-2-enoic acid[4-(3-chloro-4-fluoro-phenylamino)-3-cyano-7-methoxy-quinolin-6-yl]-amide or a pharmaceutically acceptable salt thereof; hh) 4-(2,5-Dimethyl-pyrrolidin-1-yl)-but-2-enoic acid [4-(3-chloro-4-fluoro-phenylamino)-3-cyano-7-methoxy-quinolin-6-yl]-amide or a pharmaceutically acceptable salt thereof; ii) 4-(4,4-Dihydroxy-piperidin-1-yl)-but-2-enoic acid[4-(3-chloro-4-fluoro-phenylamino)-3-cyano-7-methoxy-quinolin-6-yl]-amide or a pharmaceutically acceptable salt thereof; jj) 4-(3-Chloro-4-fluoro-phenylamino)-7-methoxy-6-pyrrolidin-1-yl-quinoline-3-carbonitrile or a pharmaceutically acceptable salt thereof; kk) 4-(3-Chloro-4-fluoroanilino)-7-methoxy-6-(1H-pyrrol-1-yl)-3-quinolinecarbonitrile or a pharmaceutically acceptable salt thereof; ll) 6-(1-Aziridinyl)-4-(3-chloro-4-fluoroanilino)-7-methoxy-3-quinolinecarbonitrile or a pharmaceutically acceptable salt thereof; mm) 4-[(2-Methoxy-ethyl)-methyl-amino]-but-2-enoic acid [4-(3-bromo-phenylamino)-3-cyano-7-ethoxy-quinolin-6-yl]-amide or a pharmaceutically acceptable salt thereof; nn) 4-(2,4-Dichloro-5-methoxy-phenylamino)-7-[3-(4-hydroxy-piperidin-1-yl)-propoxy]-6-methoxy-quinoline-3-carbonitrile or a pharmaceutically acceptable salt thereof; oo) 4-(2,4-Dichloro-5-methoxy-phenylamino)-7-{3-[4-(2-hydroxy-ethyl)-piperazin-1-yl]-propoxy}-6-methoxy-quinoline-3-carbonitrile or a pharmaceutically acceptable salt thereof; pp) 4-(2-Bromo-4-chloro-phenylamino)-7-{2-[(2-hydroxy-ethyl)-methyl-amino]-ethoxy}-6-methoxy-quinoline-3-carbonitrile or a pharmaceutically acceptable salt thereof; qq) 4-(2,4-Dichloro-5-methoxy-phenylamino)-7-{3-[(2-hydroxy-ethyl)-methyl-amino]-propoxy}-6-methoxy-quinoline-3-carbonitrile or a pharmaceutically acceptable salt thereof; rr) 4-(2,4-Dichloro-5-methoxy-phenylamino)-6-methoxy-7-(3-thiomorpholin-4-yl-propoxy)-quinoline-3-carbonitrile or a pharmaceutically acceptable salt thereof; ss) 4-(2,4-Dichloro-5-methoxy-phenylamino)-6-methoxy-7-[3-(2-methoxy-ethylamino)-propoxy]-quinoline-3-carbonitrile or a pharmaceutically acceptable salt thereof; tt) 4-(2,4-Dichloro-5-methoxy-phenylamino)-6-methoxy-7-[3-(4-methyl-piperidin-1-yl)-propoxy]-quinoline-3-carbonitrile or a pharmaceutically acceptable salt thereof; uu) 4-(2,4-Dichloro-5-methoxy-phenylamino)-7-[3-(2,6-dimethyl-morpholin-4-yl)-propoxy]-6-methoxy-quinoline-3-carbonitrile or a pharmaceutically acceptable salt thereof; vv) 4-(2-Bromo-4-chloro-phenylamino)-7-{2-[4-(2-hydroxy-ethyl)-piperazin-1-yl]-ethoxy}-6-methoxy-quinoline-3-carbonitrile or a pharmaceutically acceptable salt thereof; ww) 4-(2-Bromo-4-chloro-phenylamino)-7-[2-(4-hydroxy-piperidin-1-yl)-ethoxy]-6-methoxy-quinoline-3-carbonitrile or a pharmaceutically acceptable salt thereof; xx) 4-(2-Bromo-4-chloro-phenylamino)-6-methoxy-7-(2-thiomorpholin-4-yl-ethoxy)-quinoline-3-carbonitrile or a pharmaceutically acceptable salt thereof; yy) 4-(2,4-Dichloro-5-methoxy-phenylamino)-7-[3-(2,5-dimethyl-pyrrolidin-1-yl)-propoxy]-6-methoxy-quinoline-3-carbonitrile or a pharmaceutically acceptable salt thereof; zz) 4-(2,4-Dichloro-5-methoxy-phenylamino)-7-[3-(3-hydroxy-propylamino)-propoxy]-6-methoxy-quinoline-3-carbonitrile or a pharmaceutically acceptable salt thereof; aaa) 1-{3-[3-Cyano-4-(2,4-dichloro-5-methoxy-phenylamino)-6-methoxy-quinolin-7-yloxy]-propyl}-piperidine-4-carboxylic acid ethyl ester or a pharmaceutically acceptable salt thereof; bbb) 7-[3-(4-acetyl-1-piperazinyl)propoxy]-4-[(2,4-dichloro-5-methoxyphenyl)amino]-6-methoxy-3-quinolinecarbonitrile or a pharmaceutically acceptable salt thereof; ccc) 4-(3-chloro-4-fluoroanilino)-7-methyoxy-6(4-morpholinyl)-3-quinolinecarbonitrile or a pharmaceutically acceptable salt thereof; ddd) 7-[3-(4-Benzyl-piperazin-1-yl)-propoxy]-4-(2,4-dichloro-5-methoxy-phenylamino)-6-methoxy-quinoline-3-carbonitrile or a pharmaceutically acceptable salt thereof; eee) 4-(2,4-Dichloro-5-methoxy-phenylamino)-7-[3-(2-hydroxy-ethylamino)-propoxy]-6-methoxy-quinoline-3-carbonitrile or a pharmaceutically acceptable salt thereof; fff) 4-(2,4-Dichloro-5-methoxy-phenylamino)-7-{3-[ethyl-(2-hydroxy-ethyl)-amino]-propoxy}-6-methoxy-quinoline-3-carbonitrile or a pharmaceutically acceptable salt thereof; ggg) 7-{3-[Bis-(2-methoxy-ethyl)-amino]-propoxy}-4-(2,4-dichloro-5-methoxy-phenylamino)-6-methoxy-quinoline-3-carbonitrile or a pharmaceutically acceptable salt thereof; hhh) 7-{3-[Bis-(2-hydroxy-ethyl)-amino]-propoxy}-4-(2,4-dichloro-5-methoxy-phenylamino)-6-methoxy-quinoline-3-carbonitrile or a pharmaceutically acceptable salt thereof; iii) 4-(3-chloro-4-fluoroanilino)-7-(4-morpholinyl)-6-nitro-3-quinolinecarbonitrile or a pharmaceutically acceptable salt thereof; jjj) N-[4-(3-chloro-4-fluoroanilino)-3-cyano-7-(4-morpholinyl)-6-quinolinyl]-2-butynamide or a pharmaceutically acceptable salt thereof; kkk) 6-amino-4-(3-chloro-4-fluoroanilino)-7-(4-morpholinyl)-3-quinolinecarbonitrile or a pharmaceutically acceptable salt thereof; lll) 4-(2,4-dichloro-5-methoxyanilino)-6-methoxy-7-(3-{[2-(4-morpholinyl)ethyl]amino} propoxy)-3-quinolinecarbonitrile or a pharmaceutically acceptable salt thereof; mmm) 7-{3-[(2-anilinoethyl)amino]propoxy}-4-(2,4-dichloro-5-methoxyanilino)-6-methoxy-3-quinolinecarbonitrile or a pharmaceutically acceptable salt thereof; nnn) N-[4-(3-chloro-4-fluoroanilino)-3-cyano-7-(4-morpholinyl)-6-quinolinyl]acrylamide or a pharmaceutically acceptable salt thereof; ooo) 4-(3-chloro-4-fluoroanilino)-7-{4-[2-(dimethylamino) ethyl]-1-piperazinyl}-6-nitro-3-quinolinecarbonitrile or a pharmaceutically acceptable salt thereof; ppp) 6-amino-4-(3-chloro-4-fluoroanilino)-7-{4-[2-(dimethylamino)ethyl]-1-piperazinyl}-3-quinolinecarbonitrile or a pharmaceutically acceptable salt thereof; qqq) N-(4-(3-chloro-4-fluoroanilino)-3-cyano-7-{4-[2-(dimethylamino)ethyl]-1-piperazinyl}-6-quinolinyl)acrylamide or a pharmaceutically acceptable salt thereof; rrr) 4-(2,4-dichloro-5-methoxyanilino)-6-methoxy-7-({2-[4-(2-methoxyethyl)-1-piperazinyl]ethyl}amino)-3-quinolinecarbonitrile or a pharmaceutically acceptable salt thereof; sss) 4-(2,4-dichloro-5-methoxyanilino)-6-methoxy-7-[3-(2H-1,2,3-triazol-2-yl)propoxy]-3-quinolinecarbonitrile or a pharmaceutically acceptable salt thereof; ttt) 4-(2,4-dichloro-5-methoxyanilino)-6-methoxy-7-[3-(1H-1,2,3-triazol-1-yl)propoxy]-3-quinolinecarbonitrile or a pharmaceutically acceptable salt thereof; uuu) 4-(2,4-dichloro-5-methoxyanilino)-6-methoxy-7-(3-thienyl)-3-quinolinecarbonitrile or a pharmaceutically acceptable salt thereof; vvv) 4-[(E)-2-(2-quinolinyl)ethenyl]aniline or a pharmaceutically acceptable salt thereof; www) 4-(2,4-dichloro-5-methoxyanilino)-6-methoxy-7-{[2-(2H-1,2,3-triazol-2-yl)ethyl]amino}-3-quinolinecarbonitrile or a pharmaceutically acceptable salt thereof; xxx) 4-(2,4-dichloro-5-methoxyanilino)-6-methoxy-7-{[2-(1H-1,2,3-triazol-1-yl)ethyl]amino}-3-quinolinecarbonitrile or a pharmaceutically acceptable salt thereof; yyy) 4-(2,4-dichloro-5-methoxyanilino)-7-(3-thienyl)-3-quinolinecarbonitrile or a pharmaceutically acceptable salt thereof; zzz) 4-(2,4-dichloro-5-methoxyanilino)-6-methoxy-7-[3-(1H-1,2,4-triazol-1-yl)propoxy]-3-quinolinecarbonitrile or a pharmaceutically acceptable salt thereof; aaaa) 4-(2,4-dichloro-5-methoxyanilino)-7-[3-(1H-imidazol-1-yl)propoxy]-6-methoxy-3-quinolinecarbonitrile or a pharmaceutically acceptable salt thereof; bbbb) 4-(2,4-dichloro-5-methoxyanilino)-6-methoxy-7-[3-(1H-pyrazol-1-yl)propoxy]-3-quinolinecarbonitrile or a pharmaceutically acceptable salt thereof; cccc) N-[3-cyano-4-(2,4-dichloro-5-methoxyanilino)-6-methoxy-7-quinolinyl]-N-[4-(4-ethyl-1-piperazinyl)butyl]acetamide or a pharmaceutically acceptable salt thereof; dddd) N-[3-cyano-4-(2,4-dichloro-5-methoxyanilino)-6-methoxy-7-quinolinyl]-N-(3-(4-ethyl-1-piperazinyl)propyl)acetamide or a pharmaceutically acceptable salt thereof; eeee) 4-(2,4-dichloro-5-methoxyanilino)-6-methoxy-7-{3-[4-(2-methoxyethyl)-1-piperazin yl]propoxy}-3-quinolinecarbonitrile or a pharmaceutically acceptable salt thereof; ffff) 4-(2,4-dichloro-5-methoxyanilino)-6-methoxy-7-(1H-pyrrol-1-yl)-3-quinolinecarbonitrile or a pharmaceutically acceptable salt thereof; gggg) 4-(4-bromo-2-fluoroanilino)-6-methoxy-7-[2-(1H-1,2,3-triazol-1-yl)ethoxy]-3-quinolinecarbonitrile or a pharmaceutically acceptable salt thereof; hhhh) 4-(4-bromo-2-fluoroanilino)-6-methoxy-7-[2-(2H-1,2,3-triazol-2-yl)ethoxy]-3-quinolinecarbonitrile or a pharmaceutically acceptable salt thereof; iiii) 4-(2,4-dichloro-5-methoxyanilino)-6-methoxy-7-[3-(1H-tetraazol-1-yl)propoxy]-3-quinolinecarbonitrile or a pharmaceutically acceptable salt thereof; jjjj) 4-(2,4-dichloro-5-methoxyanilino)-6-methoxy-7-[3-(2H-tetraazol-2-yl)propoxy]-3-quinolinecarbonitrile or a pharmaceutically acceptable salt thereof; kkkk) 4-(4-bromo-2-fluoroanilino)-6-methoxy-7-[2-(1H-1,2,3-triazol-1-yl)ethoxy]-3-quinolinecarbonitrile or a pharmaceutically acceptable salt thereof; llll) 4-(4-bromo-2-fluoroanilino)-6-methoxy-7-[2-(2H-1,2,3-triazol-2-yl)ethoxy]-3-quinolinecarbonitrile or a pharmaceutically acceptable salt thereof; or mmmm) 4-(2,4-dichloro-5-methoxyanilino)-7-{3-[[2-(dimethylamino)ethyl](methyl)amino]propoxy}-6-methoxy-3-quinolinecarbonitrile or a pharmaceutically acceptable salt thereof.

Other suitable examples of RTK inhibitors include, but are not limited to compounds selected from: 4-({3-Chloro-4-[(1,4,5-trimethyl-1H-imidazol-2-yl)thio]phenyl}amino-7-{[3-(dimethylamino)propyl]amino}-6-methoxyquinoline-3-carbonitrile, 4-({4-[(1-benzyl-4,5-dimethyl-1H-imidazol-2-yl)thio]-3-chloro-phenyl}amino)-7-{[3-(dimethylamino)propyl]amino}-6-methoxyquinoline-3-carbonitrile, 4-({3-chloro-4-[(1,5-dimethyl-1H-benzimidazol-2-yl)thio]phenyl}amino)-7-{[3-(dimethylamino)propyl]amino}-6-methoxyquinoline-3-carbonitrile, 4-({3-chloro-4-[(1-ethyl-4,5-dimethyl-1H-imidazol-2-yl)thio]phenyl}amino)-7-{[3(dimethylamino)-propyl]amino}-6-methoxyquinoline-3-carbonitrile, 4-({3-bromo-4-[(1,4,5-trimethyl-1H-imidazol-2-yl)thio]phenyl}amino)-7-{[3-(dimethylamino)-propyl]amino}-6-methoxy-quinoline-3-carbonitrile, 7-{[3-(dimethyl-amino)propyl]-amino}-6-methoxy-4-({4-[(1,4,5-trimethyl-1H imidazol-2-yl)thio]phenyl}amino)-quinoline-3-carbonitrile, 4-({3-Chloro-4-[(1-ethyl-4,5-dimethyl-1H-imidazol-2-yl)thio]phenyl}-amino)-6-methoxy-7-[(3-phenylpropyl)amino]quinoline-3-carbonitrile, 4-({3-chloro-4-[(1-ethyl-4,5-dimethyl-1H-imidazol-2-yl)thio]phenyl}-amino)-6-methoxy-7-[(3-morpholin-4-ylpropyl)amino]-quinoline-3-carbonitrile, 7-({3-[bis(2-hydroxyethyl)amino]propyl}amino)-4-({3-chloro-4-[(1-ethyl-4,5-dimethyl-1H-imidazol-2-yl)thio]phenyl}amino)-6-methoxyquinoline-3-carbonitrile, N-(3-{4-[3-chloro-4-(1-ethyl-4,5-dimethyl-1H-imidazol-2-ylsulfanyl)-phenylamino]-3-cyano-6-methoxy-quinolin-7-yloxy}-propyl)-benzenesulfonamide, 7-{3-[tert-butyl(2-hydroxy-ethyl)amino]propoxy}-4-({3-chloro-4-[(1-ethyl-4,5-dimethyl-1H-imidazol-2-yl)thio]phenyl}amino)-6-methoxyquinoline-3-carbonitrile, 4-({3-chloro-4-[(1-ethyl-4,5-dimethyl-1H-imidazol-2-yl)thio]phenyl}amino)-7-[3-(dimethyl-amino)propoxy]-6-methoxyquinoline-3-carbonitrile, ethyl 4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperazine-1-carboxylate, 4-({3-chloro-4-[(1-ethyl-4,5-dimethyl-1H-imidazol-2-yl)thio]phenyl}amino)-7-[3-(2-ethylpiperidin-1-yl)propoxy]-6-methoxy-quinoline-3-carbonitrile, ethyl 1-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-piperidine-4-carboxylate, N-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)-methanesulfonamide, 4-({3-Chloro-4-[(1-ethyl-4,5-dimethyl-1H-imidazol-2-yl)sulfanyl]phenyl}amino)-7-[3-(4-ethyl-1-piperazinyl)-propoxy]-6-methoxy-3-quinolinecarbonitrile, 4-({3-chloro-4-[(1-ethyl-4,5-dimethyl-1H-imidazol-2-yl)thio]-phenyl}amino)-7-{3-[4-(2-hydroxyethyl)piperazin-1-yl]propoxy}-6-methoxy-quinoline-3-carbonitrile, 4-({3-chloro-4-[(1-ethyl-4,5-dimethyl-1H-imidazol-2-yl)thio]phenyl}amino)-7-{3-[(2-hydroxyethyl)(methyl)amino]propoxy}-6-methoxy-quinoline-3-carbonitrile, 4-({3-chloro-4-[(1-ethyl-4,5-dimethyl-1H-imidazol-2-yl)thio]phenyl}amino)-7-[3-(3-hydroxypyrrolidin-1-yl)propoxy]-6-methoxyquinoline-3-carbonitrile, 4-({3-chloro-4-[(1-ethyl-4,5-dimethyl-1H-imidazol-2-yl)thio]phenyl}amino)-6-methoxy-7-(3-pyrrolidin-1-ylpropoxy)quinoline-3-carbonitrile, ethyl [4-(3-{[4-({3-chloro-4-[(1-ethyl-4,5-dimethyl-1H-imidazol-2-yl)thio]phenyl}amino)-3-cyano-6-methoxyquinolin-7-yl]oxy}propyl)piperazin-1-yl]acetate, 4-({3-chloro-4-[(1-ethyl-4,5-dimethyl-1H-imidazol-2-yl)thio]phenyl}amino)-7-{3-[(2,3-dihydroxypropyl)(methyl)-amino]propoxy}-6-methoxyquinoline-3-carbonitrile, 4-({3-chloro-4-[(1-ethyl-4,5-dimethyl-1H-imidazol-2-yl)thio]phenyl}amino)-6-methoxy-7-(3-morpholin-4-ylpropoxy)quinoline-3-carbonitrile, 4-({3-chloro-4-[(1-ethyl-4,5-dimethyl-1H-imidazol-2-yl)thio]phenyl}amino)-7-{3-[[2-(1,3-dioxolan-2-yl)ethyl](methyl)amino]propoxy}-6-methoxyquinoline-3-carbonitrile, 4-({3-chloro-4-[(1-ethyl-4,5-dimethyl-1H-imidazol-2-yl)thio]phenyl}amino)-7-[3-(4-hydroxypiperidin-1-yl)propoxy]-6-methoxyquinoline-3-carbonitrile, 7-[3-(4-acetylpiperazin-1-yl)propoxy]-4-({3-chloro-4-[(1-ethyl-4,5-dimethyl-1Himidazol-2-yl)thio]phenyl}amino)-6-methoxyquinoline-3-carbonitrile, 4-[3-Chloro-4-(1-ethyl-4,5-dimethyl-1H-imidazol-2-ylsulfanyl)-phenylamino]-6-methoxy-7-[3-(4-methyl-piperazin-1-yl)-propylamino]-quinoline-3-carbonitrile, 4-[3-Chloro-4-(4,5-dimethyl-1-propyl-1H-imidazol-2-ylsulfanyl)-phenylamino]-7-(3-dimethylamino-propylamino)-6-methoxy-quinoline-3-carbonitrile, 4-[3-Chloro-4-(1-isopropyl-4,5-dimethyl-1H-imidazol-2-ylsulfanyl)-phenylamino]-7-(3-dimethylamino-propylamino)-6-methoxy-quinoline-3-carbonitrile and pharmaceutically acceptable salts thereof.

Where present, RTK inhibitors, including EGFR inhibitors, of the invention and corresponding pharmaceutically acceptable salts or esters thereof include isomers either individually or as a mixture, such as enantiomers, diastereomers, and positional isomers. “Pharmaceutically acceptable salts and esters” refers to salts and esters that are pharmaceutically acceptable and have the desired pharmacological properties. Such salts include, for example, salts that can be formed where acidic protons present in the compounds are capable of reacting with inorganic or organic bases. Suitable inorganic salts include, for example, those formed with the alkali metals or alkaline earth metals, e.g. sodium and potassium, magnesium, calcium, and aluminum. Suitable organic salts include, for example, those formed with organic bases such as the amine bases, e.g. ethanolamine, diethanolamine, triethanolamine, tromethamine, N-methylglucamine, and the like. Pharmaceutically acceptable salts can also include acid addition salts formed from the reaction of basic moieties, such as amines, in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid).

Pharmaceutically acceptable esters include esters formed from carboxy, sulfonyloxy, and phosphonoxy groups present in the compounds of the invention, e.g. C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

Pharmaceutically acceptable salts of RTK inhibitors, including EGFR inhibitors, of the invention with an acidic moiety may be formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be formed from organic and inorganic acids. For example salts may be formed from acids: acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when a compound of this invention contains a basic functional group. Other suitable examples of pharmaceutically acceptable salts include, but are not limited, to sulfate; citrate, acetate; oxalate; chloride; bromide; iodide; nitrate; bisulfate; phosphate; acid phosphate; isonicotinate; lactate; salicylate; acid citrate; tartrate; oleate; tannate; pantothenate; bitartrate; ascorbate; succinate; maleate; gentisinate; fumarate; gluconate; glucaronate; saccharate; formate; benzoate; glutamate; methanesulfonate; ethanesulfonate; benzenesulfonate; p-toluenesulfonate; pamoate (i.e., 1,1′-methylene-bis-(2-hydroxy-3-naphthoate)); and salts of fatty acids such as caproate, laurate, myristate, palmitate, stearate, oleate, linoleate, and linolenate salts. The compounds can also be used in the form of esters, carbamates and other conventional ester forms, also reffered to herein as prodrug forms, which when administered in such form, convert to the active moiety in-vivo. Exemplary ester forms of the compounds of this invention include, but are not limited to, straight chain alkyl esters having from 1 to 6 carbon atoms or branched chain alkyl groups containing 1 to 6 carbon atoms, including methyl, ethyl, propyl, butyl, 2-methylpropyl and 1,1-dimethylethyl esters, cycloalkyl esters, alkylaryl esters, benzyl esters, and the like.

As described hereinbefore, the combination of the present invention is useful in the synergistic treatment or prophylaxis of cancer. According to the present invention, a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with a RTK inhibitor or a FAS inhibitor alone. Further, the effect of the combination treatment is synergistic if a beneficial effect is obtained in a group of patients that does not respond (or responds poorly) to a RTK inhibitor or a FAS inhibitor alone. In addition, the effect of the combination treatment is defined as affording a synergistic effect if one of the components is dosed at its conventional dose and the other component is dosed at a reduced dose and the therapeutic effect, as measured by, for example, the extent of the response, the response rate, the time to disease progression or the survival period, is equivalent to that achievable on dosing conventional amounts of either one of the components of the combination treatment. In particular, synergy is deemed to be present if the conventional dose of the RTK inhibitor or FAS inhibitor may be reduced without detriment to one or more of the extent of the response, the response rate, the time to disease progression and survival data, in particular without detriment to the duration of the response, but with fewer and/or less troublesome side-effects than those that occur when conventional doses of each component are used. In one embodiment, the receptor tyrosine kinase inhibitor is an inhibitor of EGFR, namely an inhibitor of ErbB.

Cancers that are amenable to treatment with the combination product of the present invention include ovarian cancer, breast cancer, oesophageal cancer, myeloma, hepatocellular, pancreatic and cervical cancer, Ewings tumour, neuroblastoma, kaposis sarcoma, colorectal cancer, prostate cancer, bladder cancer, melanoma, lung cancer [including non small cell lung cancer (NSCLC) and small cell lung cancer (SCLC)], gastric cancer, head and neck cancer, brain cancer, renal cancer, lymphoma and leukaemia. More particularly, the combination of the present invention is useful in the treatment or prevention of ovarian cancer.

The cancer treatment of the present invention includes an anti-tumour effect that may be assessed by conventional means such as the response rate, the time to disease progression and/or the survival rate. Anti-tumour effects of the present invention include, but are not limited to, inhibition of tumour growth, tumour growth delay, regression of tumour, shrinkage of tumour, increased time to regrowth of tumour on cessation of treatment and slowing of disease progression. For example, it is expected that when the combination of the present invention is administered to a warm-blooded animal such as a human, in need of treatment for cancer involving a solid tumour, such a method of treatment will produce an effect, as measured by, for example, one or more of: the extent of the anti-tumour effect, the response rate, the time to disease progression and the survival rate.

According to one embodiment of the invention, there is provided a combination product comprising: 4-Dimethylamino-but-2-enoic acid [4-(3-chloro-4-fluoro-phenylamino)-3-cyano-7-ethoxy-quinolin-6-yl]-amide, or a pharmaceutically acceptable salt thereof, and tetrahydro-3-methylene-2-oxo-5-n-octyl-4-furancarboxylic acid.

According to a separate embodiment of the invention, there is provided a combination product comprising: (E)-N-{4-[3-chloro-4-(2-pyridinyl methoxy) anilino]-3-cyano-7-ethoxy-6-quinolinyl}-4-(dimethylamino)-2-butenamide, or a pharmaceutically acceptable salt thereof, and tetrahydro-3-methylene-2-oxo-5-n-octyl-4-furancarboxylic acid.

According to a separate embodiment of the invention, there is provided a combination product comprising: (E)-N-(4-{3-chloro-4-[(3-fluorobenzyl)oxy]anilino}-3-cyano-7-ethoxy-6-quinolinyl)-4-(dimethylamino)-2-butenamide, or a pharmaceutically acceptable salt thereof, and tetrahydro-3-methylene-2-oxo-5-n-octyl-4-furancarboxylic acid.

According to a separate embodiment of the invention, there is provided a combination product comprising: 4-(2,4-dichloro-5-methoxyanilino)-7-{5-[(4-methyl-1-piperazinyl)methyl]-2-pyridinyl}-3-carbonitrile, or a pharmaceutically acceptable salt thereof, and tetrahydro-3-methylene-2-oxo-5-n-octyl-4-furancarboxylic acid.

The present invention accordingly provides a pharmaceutical composition, which comprises an effective amount of a compound of the present invention in combination or association with a pharmaceutically acceptable carrier. Suitable examples of pharmaceutical carriers used in accordance with the present invention include, but are not limited to, excipients, diluents, fillers, disintegrants, lubricants and other agents that can function as a carrier. The term “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable. As used herein, the term “effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a securin related disorder” includes preventing the onset of symptoms in a subject that may be predisposed to a securin related disorder but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the compounds or agents of the present invention to a subject to prevent or delay, to alleviate, or to arrest or inhibit development of the symptoms or conditions associated with RTK, including EGFR, or FAS related disorders, e.g., tumor growth associated with cancer. A skilled medical practitioner will know how to use standard methods to determine whether a patient is suffering from a disease associated with activity of a RTK or a FAS, e.g., by examining the patient and determining whether the patient is suffering from a disease known to be associated with RTK or FAS activity or by assaying for RTK or FAS levels in blood plasma or tissue of the individual suspected of suffering from an RTK or FAS related disease and comparing RTK or FAS levels in the blood plasma or tissue of the individual suspected of suffering from a RTK or FAS related disease to RTK or FAS levels in the blood plasma or tissue of a healthy individual. Increased securin levels are indicative of disease. Accordingly, the present invention provides, inter alia, methods of administering a compound of the present invention to a subject and determining RTK or FAS activity in the subject. RTKor FAS activity in the subject can be determined before and/or after administration of the compound.

A “therapeutically effective amount” or “pharmaceutically effective amount” means the amount that, when administered to a subject, produces effects for which it is administered. For example, a “therapeutically effective amount,” when administered to a subject to inhibit RTK or FAS activity, is sufficient to inhibit RTK or FAS activity. A “therapeutically effective amount,” when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other routine methods allow the clinician to select patients in need of therapy using the methods and formulations of the present invention.

The compositions described herein may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the RTK inhibitor of the combination treatment may be delivered endoscopically, intratracheally, intralesionally, percutaneously, intravenously, subcutaneously, intraperitoneally or intratumourally. In general the compositions described herein may be prepared in a conventional manner using conventional excipients or carriers that are well known in the art.

Suitable pharmaceutically-acceptable excipients or carriers for a tablet formulation include, for example, inert excipients such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl 4-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Pharmaceutical compositions for oral use may be in the form of hard gelatin capsules in which the active ingredient is mixed with an inert solid excipient, for example, calcium carbonate, calcium phosphate or kaolin, or as soft gelatin capsules in which the active ingredient is mixed with water or an oil, such as peanut oil, liquid paraffin or olive oil.

The compositions of the present invention are advantageously presented in unit dosage form a RTK or FAS inhibitor as defined hereinbefore will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form Typically, unit dosage forms will contain about 0.5 mg to 0.5 g of the RTK or FAS inhibitor.

The combination product of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% w) to 10% w of the compound of formula I, preferably 0.00001% w to 1% w, with the remainder being the excipient or excipients.

The dosages and schedules described hereinbefore may be varied according to the particular disease state and the overall condition of the patient. For example, it may be necessary or desirable to reduce the above-mentioned doses of the components of the combination treatment in order to reduce toxicity. Dosages and schedules may also vary if, in addition to a combination treatment of the present invention, one or more additional chemotherapeutic agents are used. Scheduling can be determined by the practitioner who is treating any particular patient using his professional skill and knowledge.

It will be appreciated that the pharmaceutical composition according to the present invention includes a composition comprising a RTK inhibitor as defined hereinbefore and a FASN inhibitor and a pharmaceutically-acceptable excipient or carrier. Such a composition conveniently provides the therapeutic combination product of the invention for simultaneous administration in the synergistic treatment or prophylaxis of cancer.

A pharmaceutical composition according to the present invention also includes separate compositions comprising a first composition comprising a RTK inhibitor and a pharmaceutically-acceptable excipient or carrier, and a second composition comprising a FASN inhibitor and a pharmaceutically-acceptable excipient or carrier. Such a composition conveniently provides the therapeutic combination of the invention for sequential or separate administration in the synergistic treatment or prophylaxis of cancer but the separate compositions may also be administered simultaneously.

Conveniently such a pharmaceutical composition of the invention comprises a kit comprising a first container with a suitable composition containing the RTK inhibitor and a second container with a suitable composition containing a FASN inhibitor. According to this aspect of the present invention there is provided a kit for use in the synergistic treatment or prophylaxis of cancer comprising: a) a RTK inhibitor together with a pharmaceutically-acceptable excipient or carrier, in a first unit dosage form; b) a FASN inhibitor together with a pharmaceutically-acceptable excipient or carrier, in a second unit dosage form; and c) a container for containing said first and second dosage forms.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention, e.g., anti-atherosclerotic medicaments.

In another embodiment, the compounds of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). In other cases, the preferred preparation can be a lyophilized powder which may contain, for example, any or all of the following: 1 mM-50 mM histidine, 0.1%-2% sucrose, 2%-7% mannitol, at a pH range of 4.5 to 5.5, that is combined with buffer prior to use.

For treatment purposes, the compositions or compounds disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

Having described the invention, the invention is further illustrated by the following non-limiting examples.

### EXAMPLES

### Example 1

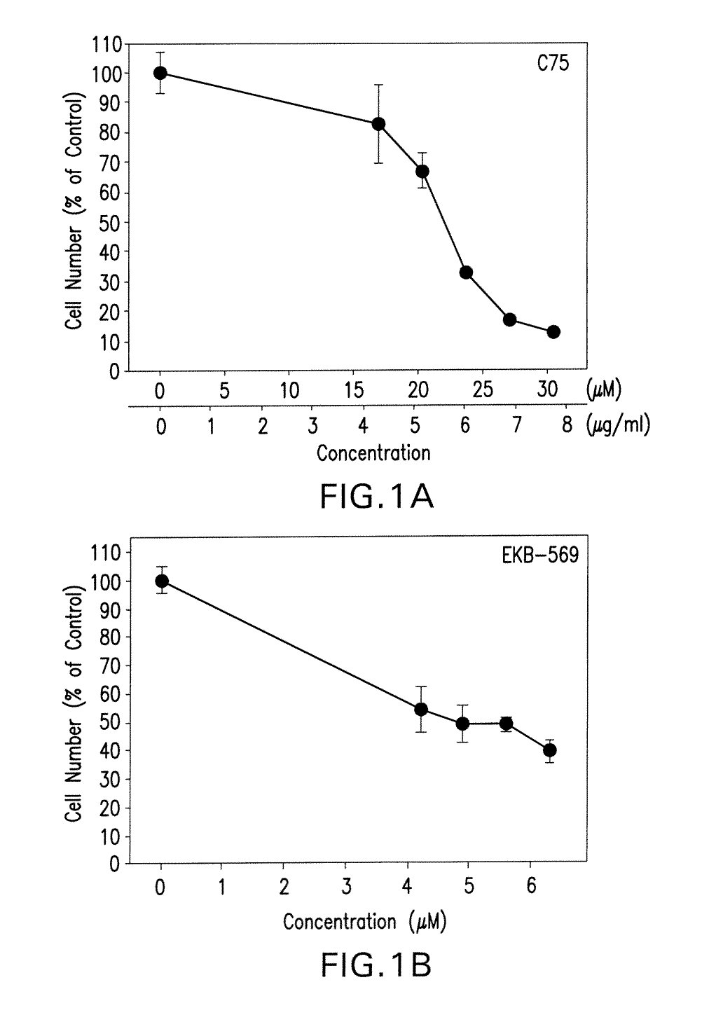

Effect of FAS and ErbB inhibition on A2780 ovarian cancer cells (OCC). A FASN inhibitor (C75) and an irreversible ErbB inhibitor (EKB-569,4-Dimethylamino-but-2-enoic acid [4-(3-chloro-4-fluoro-phenylamino)-3-cyano-7-ethoxy-quinolin-6-yl]-amide) inhibit growth of OCC (MTT assay—IC₅₀: C75=22 μM; EKB-569=5, 1 μM). A dose-dependent reduction of in-vitro cell growth of A2780 ovarian cancer cells by a combination of a synthetic FASN inhibitor (C75) and of an ErbB inhibitor (EKB-569) as demonstrated by formazan dye assay, as shown in FIG. 1. Cells were grown for three days in the presence of vehicle (0.1% DMSO) or synthetic inhibitor before cell number was estimated. Data in the charts represent means +/−SD of triplicate measurements. Table 1 shows the concentrations of each individual inhibitor required for 50% reduction of cell growth (IC₅₀-values, means +/−SD of 3 to 5 independent experiments).

TABLE 1
IC₅₀-values for a combination product
(C75 + EKB-569) in OCC study.

| | IC50 (μM) |
|---|---|
| Drug | Mean +/− SD* |
| C75 | 21.90 +/− 3.38 |
| Pelitinib | 5.14 +/− 1.42 |

*n = 3-5

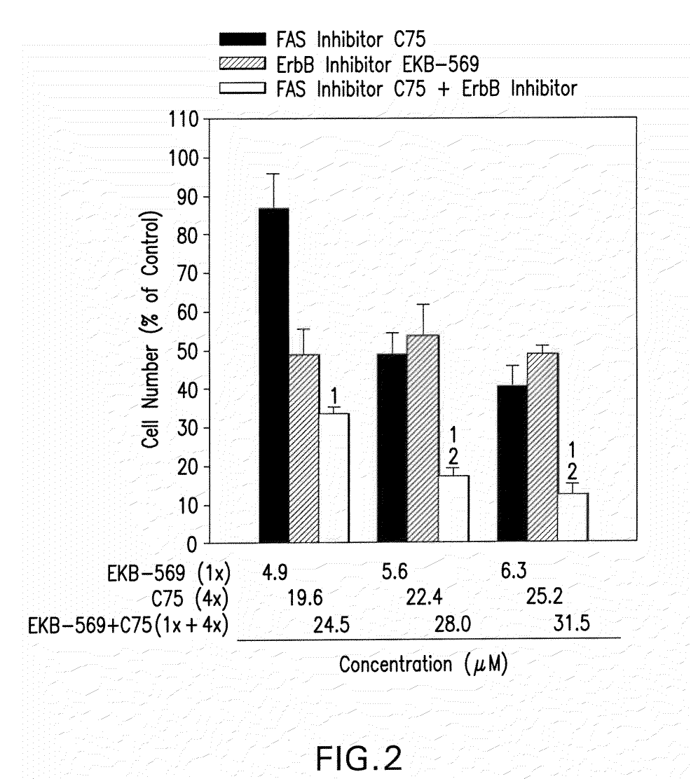

Interestingly, C75 synergizes with EKB-569 in cell growth inhibition (p<0.01) suggesting cooperation between FAS and ErbB pathways during OCC growth. Simultaneous exposure of the cells to the combination of a FASN inhibitor (C75) and the ErbB inhibitor (EKB-569) for 3 days followed by formazan dye assay, indicating that inhibition of FASN and ErbB enzyme cooperatively controls the in-vitro growth of A2780 ovarian cancer cells, as shown in FIG. 2. The concentrations of the ErbB inhibitor EKB-569 and of the FAS inhibitor C75 were around the respective IC₅₀ values as shown in the Table in FIG. 1. In co-treatment experiments, both classes of inhibitors were combined at an equi-efficient dose ratio according to the relationship between the respective IC₅₀ values, which was 1:4. Means +/−SD of 3 measurements are given. Statistical significance between co-treatment vs. FAS inhibitor alone (1, p<0.01) or between co-treatment and ErbB inhibitors alone (2, p<0.01) was assessed by ANOVA followed by Scheffe-test.

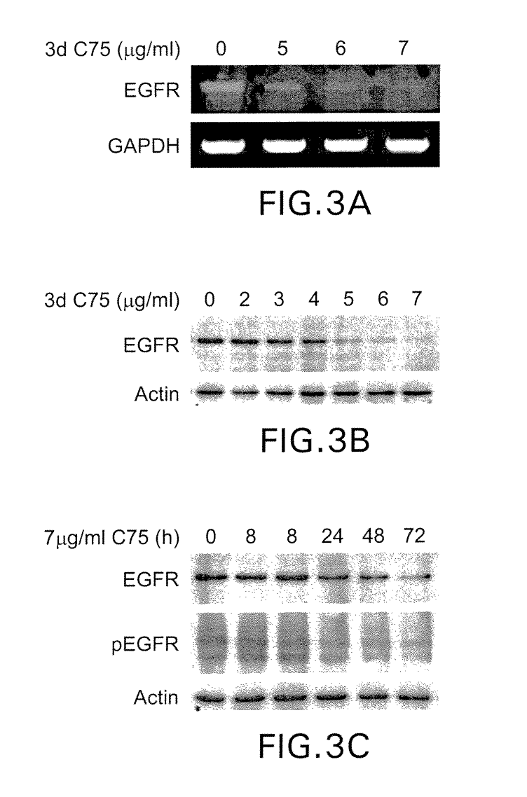

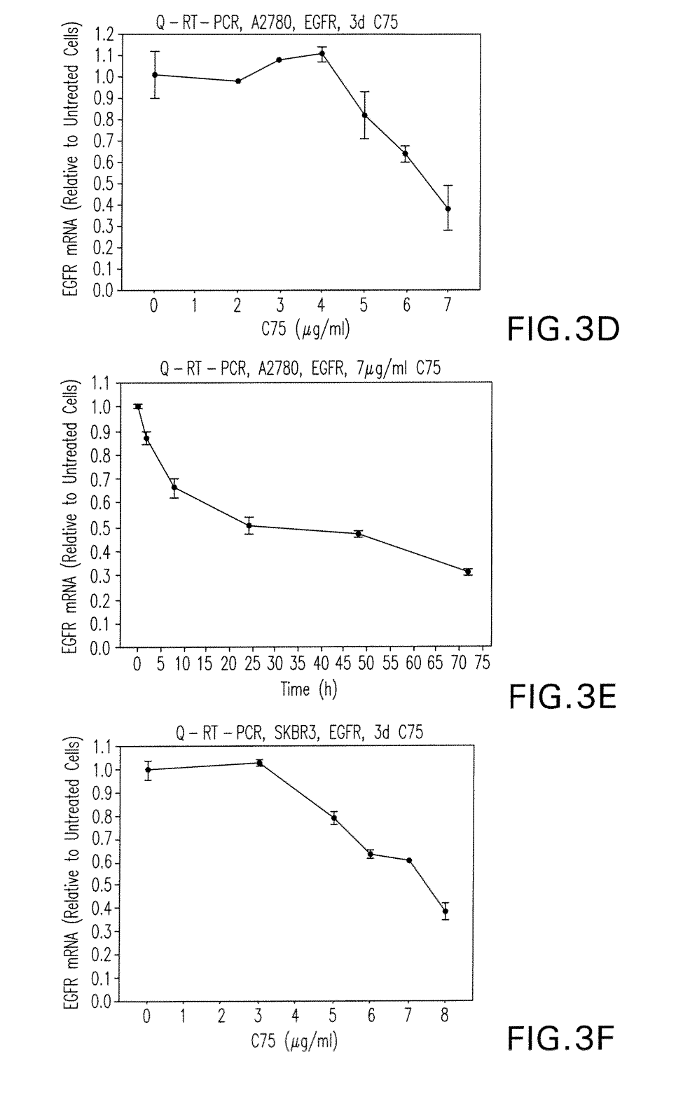

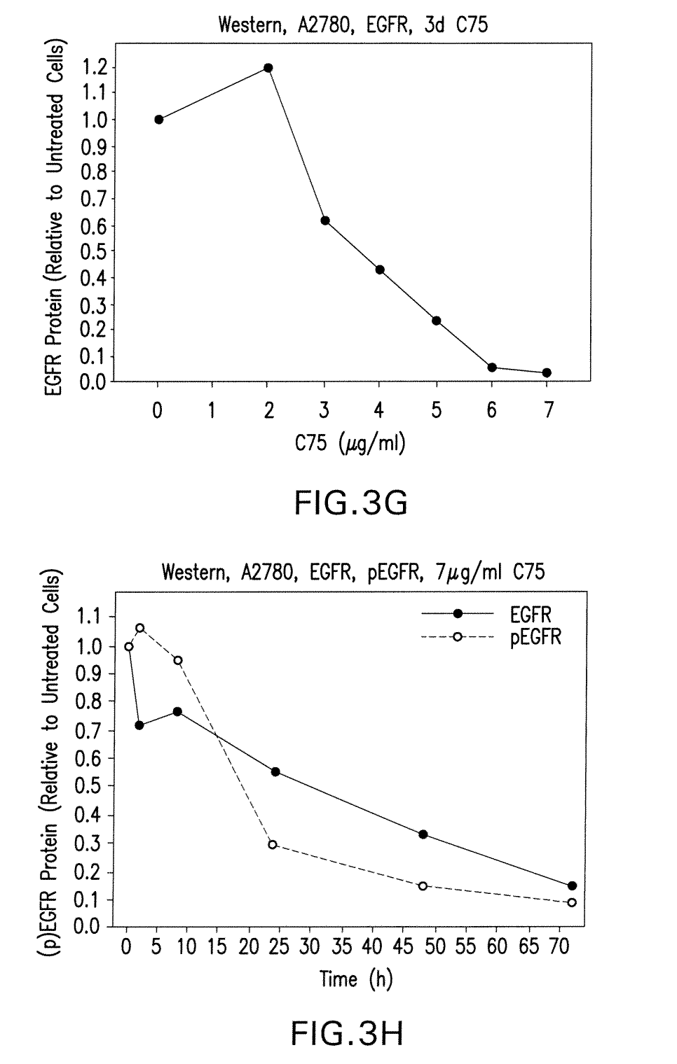

RT-PCR, real-time analysis and Western blotting revealed that C75 slowly and concordantly reduces EGFR mRNA, protein and activity in OCC. Inhibition of FASN enzyme activity by C75 down-regulates EGFR gene expression and activity in A2780 ovarian cancer cells, as shown in FIG. 3. Upper panel: RT-PCR analysis of EGFR mRNA steady-state levels in cells exposed for 3 days to C75 at the indicated concentrations. Intermediate panels: Q-RT-PCR analysis demonstrating dose-(left) and time-dependent (middle) repression of EGFR mRNA expression in A2780 cells. Intermediate right panel: SKBR-3 breast cancer cells serve as control cells and demonstrate corresponding dose-dependent down-regulation of EGFR mRNA by C75 treatment. Lower panels: Western blot analysis of C75-mediated dose-(upper) and time-dependent (lower) reduction of total and phosphorylated EGFR protein levels. Right panels: Graphical representation of densitometric quantitation of (phospho-) EGFR levels relative to actin levels.

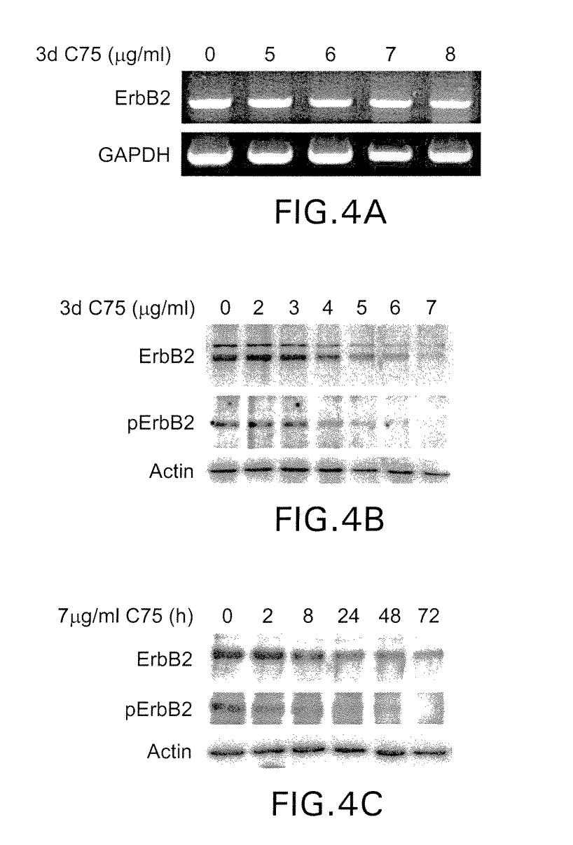

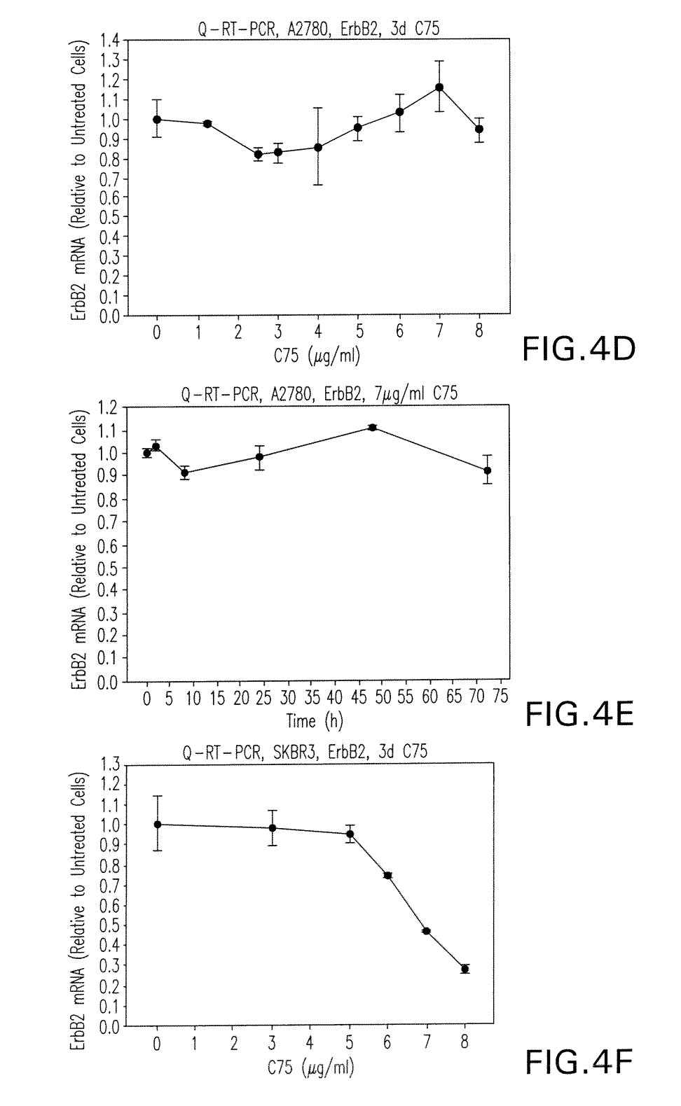

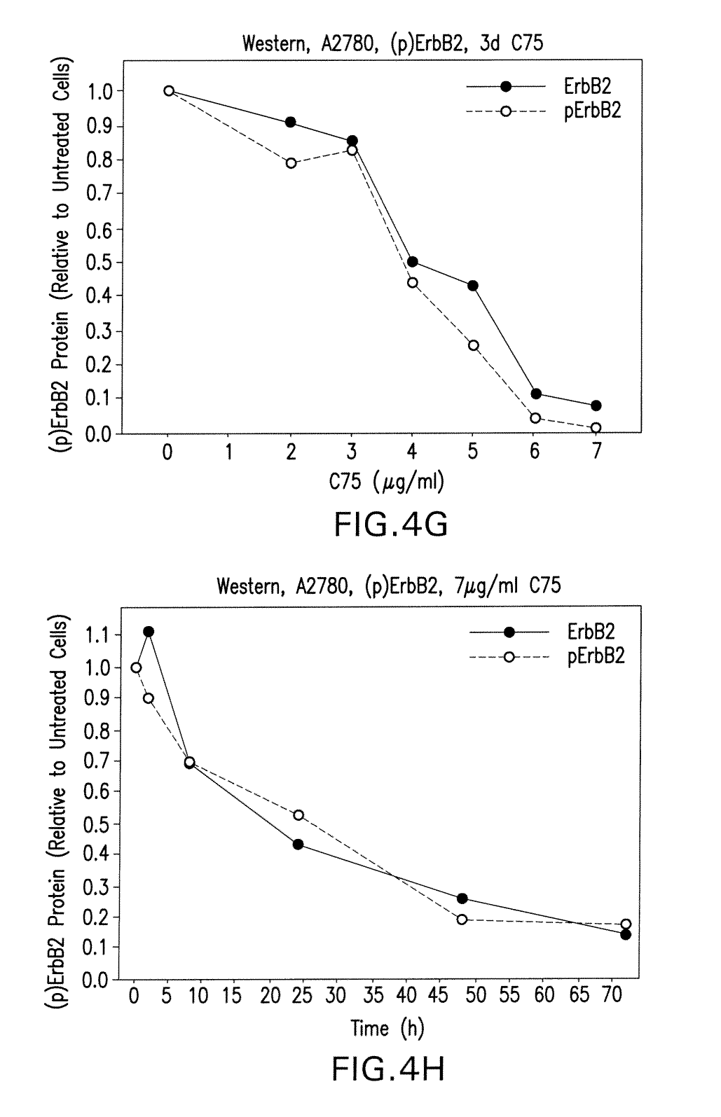

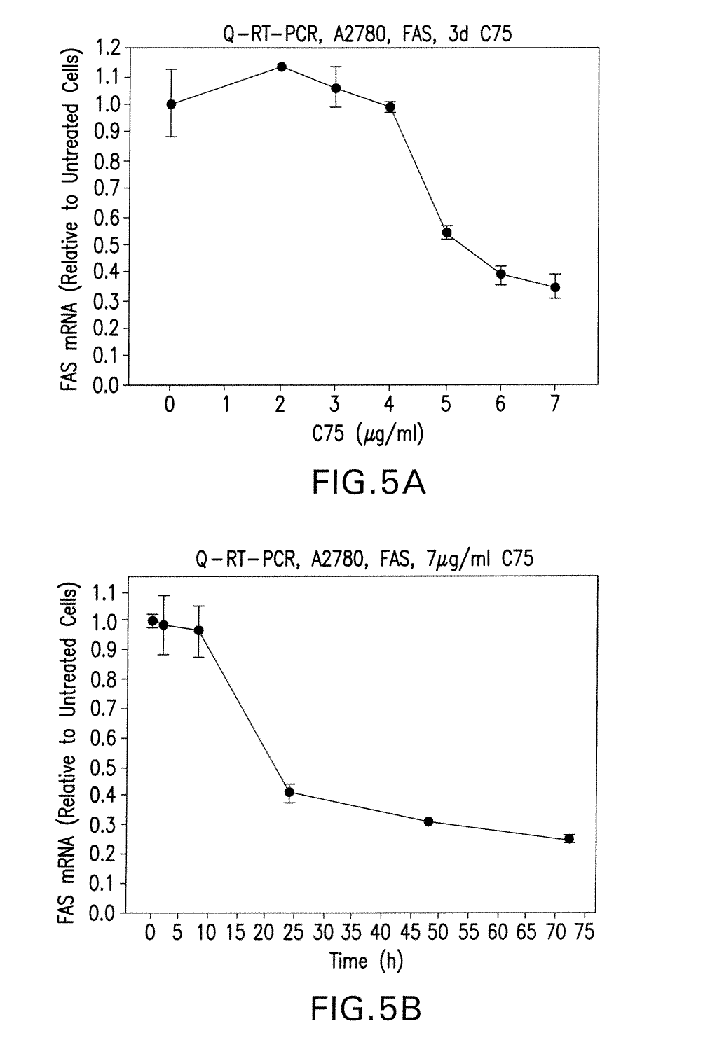

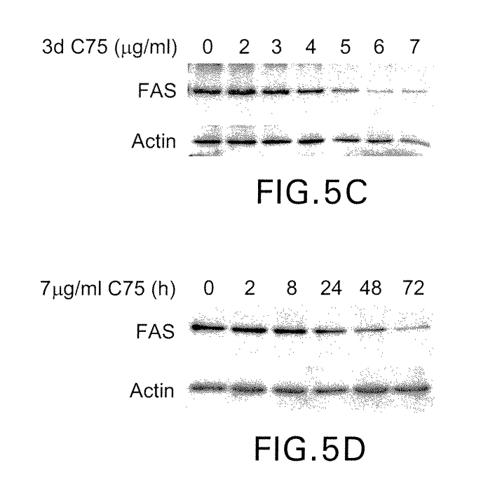

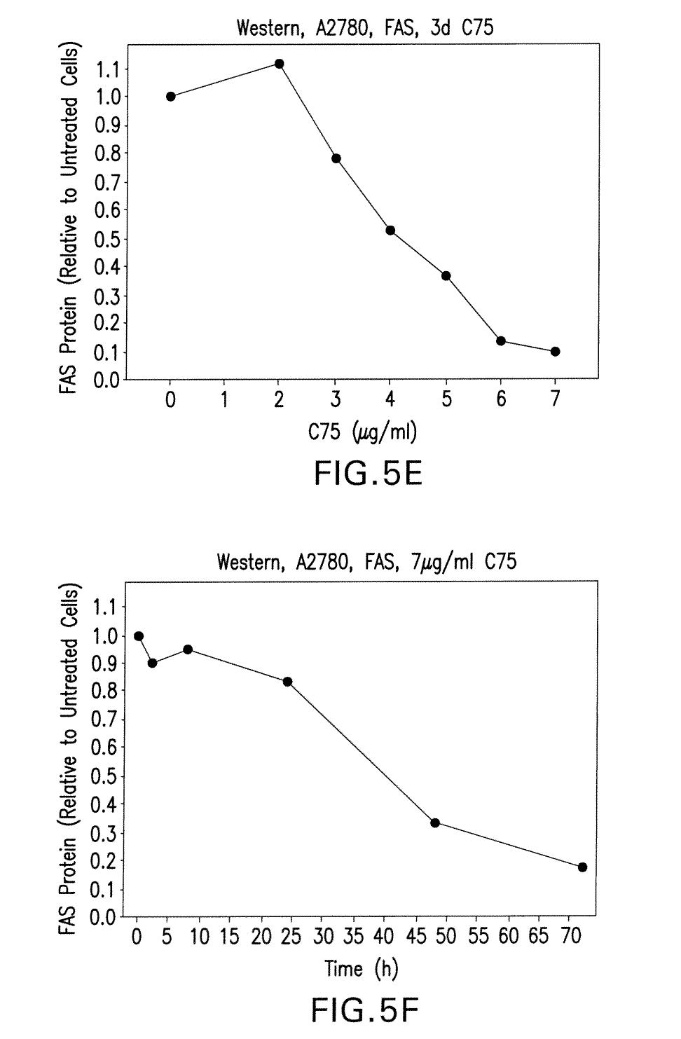

Thus, C75 silences EGFR gene expression at transcriptional levels without directly affecting EGFR signaling. C75 caused deprivation of overall and phosphorylated ErbB2 protein, but failed to diminish ErbB2 mRNA. Inhibition of FASN enzyme activity by C75 down-regulates ErbB2 protein expression and activity in A2780 ovarian cancer cells, as shown in FIG. 4. Upper panel: RT-PCR analysis of ErbB2 mRNA steady-state levels in cells exposed for 3 days to C75 at the indicated concentrations. Intermediate panels: Q-RT-PCR analysis demonstrating lack of dose-(left) and time-dependent (middle) repression of ErbB2 mRNA expression in A2780 cells. Intermediate right panel: SKBR-3 breast cancer cells serve as control cells and demonstrate dose-dependent down-regulation of ErbB2 mRNA by C75 treatment. Lower panels: Western blot analysis of C75-mediated dose-(upper) and time-dependent (lower) reduction of total and phosphorylated ErbB2 protein levels. Right panels: Graphical representation of densitometric quantitation of (phospho-) ErbB2 levels relative to actin levels.

Although C75 post-transcriptionally represses ErbB2, it does not directly disrupt ErbB2 activity. C75 also caused shut-down of FAS mRNA and protein. Inhibition of FASN enzyme activity by C75 down-regulates FASN expression levels in A2780 ovarian cancer cells, as summarized in Fugure 5. Upper panels: Q-RT-PCR analysis demonstrating dose-(left) and time-dependent (right) repression of FAS mRNA expression in A2780 cells. Lower panels: Western blot analysis of C75-mediated dose-(upper) and time-dependent (lower) reduction of FASN protein levels. Right panels: Graphical representation of densitometric quantitation of FASN protein levels relative to actin levels.

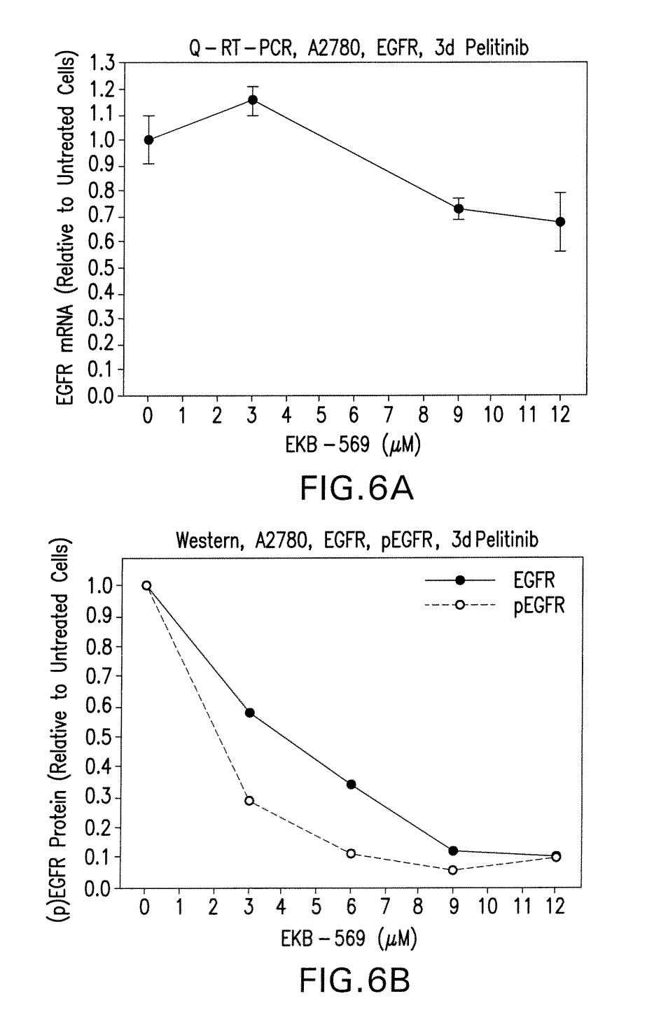

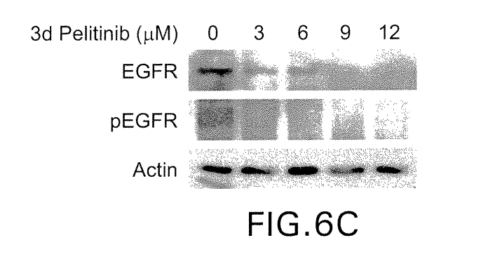

EKB-569 abolishes EGFR protein expression. Inhibition of EGFR and ErbB2 enzyme activity by EKB-569 down-regulates EGFR expression in A2780 ovarian cancer cells, as summarized in FIG. 6. Upper panel: Q-RT-PCR analysis demonstrating weak dose-dependent repression of EGFR mRNA expression in A2780 cells. Lower left panel: Western blot analysis of EKB-569-mediated dose-dependent reduction of total and phosphorylated EGFR protein levels. Lower right panel: Graphical representation of densitometric quantitation of total and phosphorylated EGFR levels relative to actin levels.

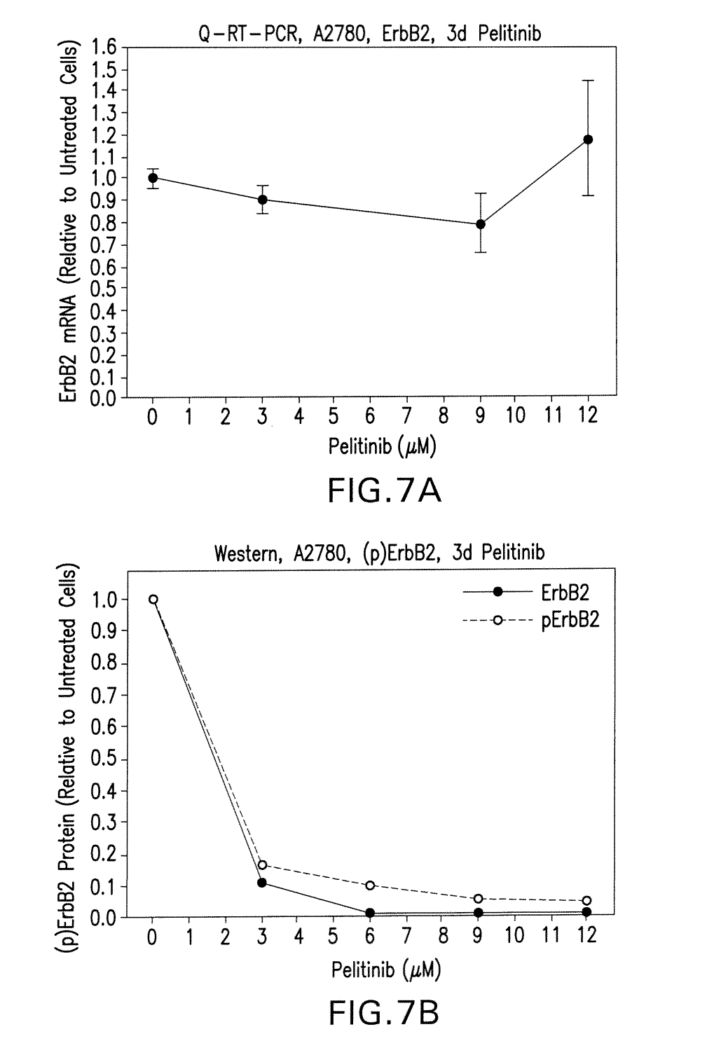

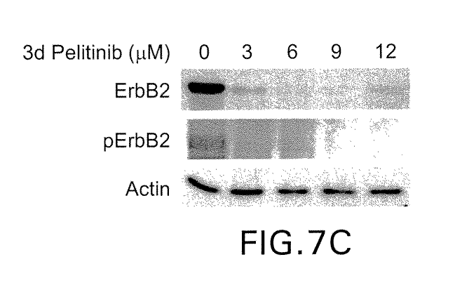

EKB-569 also abolishes ErbB2 protein expression and phosphorylation, but only weakly depresses mRNA levels. Inhibition of EGFR and ErbB2 enzyme activity by EKB-569 down-regulates ErbB2 expression in A2780 ovarian cancer cells, as shown in FIG. 7. Upper panel: Q-RT-PCR analysis demonstrating lack of dose-dependent repression of ErbB2 mRNA expression in A2780 cells. Lower left panel: Western blot analysis of EKB-569-mediated dose-dependent reduction of total and phosphorylated ErbB2 protein levels. Lower right panel: Graphical representation of densitometric quantitation of total and phosphorylated ErbB2 levels relative to actin levels.

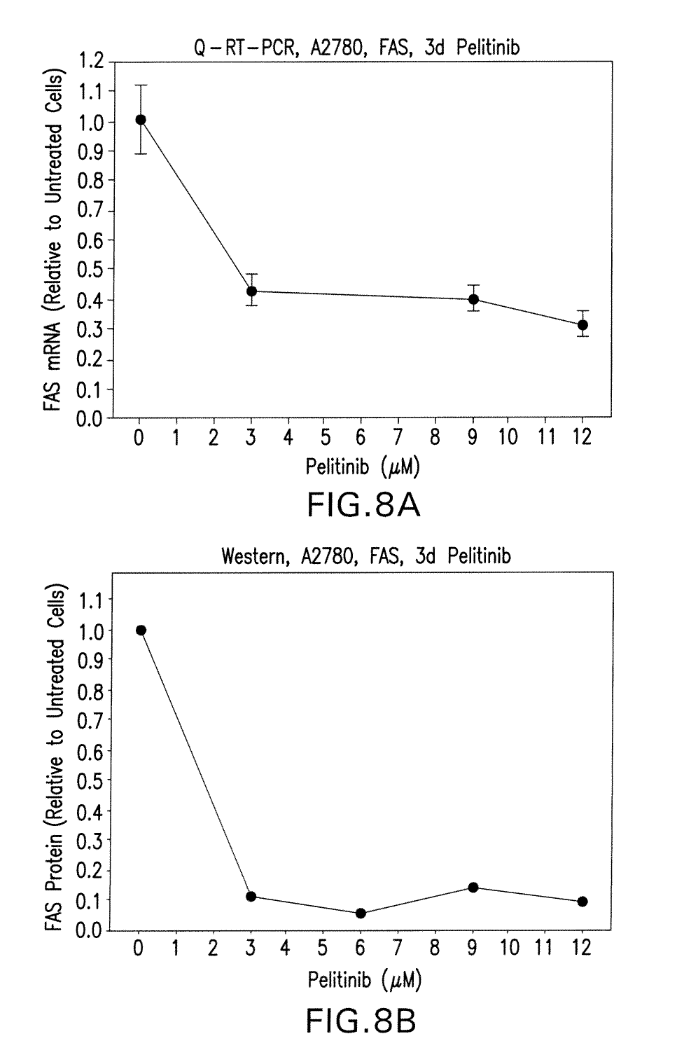

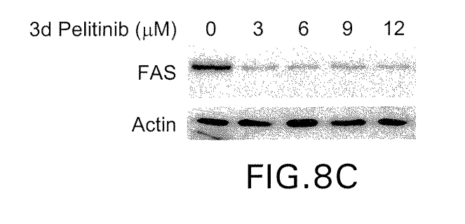

Notably, EKB-569 also represses FAS mRNA and protein. inhibition of EGFR and ErbB2 enzyme activity by EKB-569 down-regulates FASN expression in A2780 ovarian cancer cells, as summarized in FIG. 8. Upper panel: Q-RT-PCR analysis demonstrating dose-dependent repression of FAS mRNA expression in A2780 cells. Lower left panel: Western blot analysis of EKB-569-mediated dose-dependent reduction of FASN protein levels. Lower right panel: Graphical representation of densitometric quantitation of FASN levels relative to actin levels.

The results of data analysis indicate that ErbB and FAS pathways mutually interact with each other in OCC. Thus, interference with the FAS and the ErbB systems effectively abrogates their oncogenic activities and can be exploited for OCC treatment.

### Example 2

The effect of FASN and ErbB inhibition on both A2780 and SKOV3 ovarian cancer cells was examined. Concurrently contacting the cells with both a FASN inhibitor (C75), given concurrently with an ErbB inhibitory agent such as pelitinib (EKB-569), canertinib (CI-1033), erlotinib, cetuximab, matuzumab or trastuzumab, sensitizes the cells against each of the ErbB-targeting agents (p<0.01) suggesting cooperation between FASN and ErbB pathways in ovarian cancer. qRT-PCR and Western blotting revealed that C75 represses FASN mRNA and protein, and impairs EGFR, ErbB2 and AKT expression and activity, which is consistent with the notion that FASN-induced lipid rafts accommodate and stabilize ErbBs and facilitate recruitment and activation of AKT. Activated AKT negatively crosstalks with ERK and stimulates EGFR and FASN, respectively, thus feeding an autostimulatory loop, which further boosts FASN and EGFR transcription. On the other hand, pharmacologic (pelitinib, canertinib, erlotinib) or gene-specific (siRNA) targeting of EGFR and/or ErbB2 represses FASN expression. Interestingly, cotransfection of both EGFR- and ErbB2-specific siRNAs inhibits FASN expression more efficiently than either siRNA alone, indicating that both pathways have to be silenced in order to get maximal FASN downregulation. ErbB-dependent modulation of FASN is the result of PI3K/AKT/mTOR-mediated regulation of FASN mRNA transcription and protein synthesis.

### Materials and Methods

A2780 (available from the European Collection of Cell Cultures Health Protection Agency, Salisbury, Wiltshire UK) and SKOV3 (American Type Culture Collection [ATCC], Manassas, Va. US) ovarian cancer cells, and SKBR3 (ATCC) mammary carcinoma cells were maintained in RPM11640, α-MEM or DMEM, respectively, containing 10% fetal calf serum, 100 IU (μg)/ml penicillin-streptomycin and 2 mM glutamine (Gibco, Karlsruhe, Germany). The FASN inhibitor C75 (Sigma, St. Louis, Mo.) the small molecule ErbB inhibitors EKB-569 (pelitinib, Wyeth, Cambridge, Mass.), CI-1033 (canertinib, Pfizer, Groton, Conn.), and OSI-774 (erlotinib, Tarceva, Hoffmann-La Roche, Basel, Switzerland), the MEK1/2 inhibitor U0126 (Cell Signaling Technology, Boston, Mass.) and the PI3K inhibitor LY294002 (Calbiochem, San Diego, Calif.) were dissolved in dimethyl sulfoxide, stored at −80° C. in the dark and were diluted 1:10,000 or 1:1,000 in culture medium immediately before use. Aqueous stock solutions of ErbB-specific antibodies cetuximab (Erbitux, Merck, Darmstadt, Germany), matuzumab (EMD72000, Merck), and trastuzumab (Herceptin, Hoffmann-La Roche) were directly diluted to final concentration in cell-line-specific culture medium. The types and targeting features of the used anti-ErbB agents are summarized in Table 2.

TABLE 2
ErbB receptor-tyrosine kinase inhibitors

| Name | Type | ErbB Target |
|---|---|---|
| Pelitinib (EKB-569) | Irreversible TKI ¹ | EGFR, ErbB2 |
| Canertinib (CI-1033) | Irreversible TKI ¹ | EGFR, ErbB2, |
| | | ErbB4 |
| Erlotinib (OSI-774, Tarceva) | Reversible TKI ¹ | EGFR |
| Cetuximab (Erbitux) | Chimeric mAb ² | EGFR |
| Matuzumab (EMD 72000) | Humanized mAb ² | EGFR |
| Trastuzumab (Herceptin) | Humanized mAb ² | ErbB2 |

¹ TKI, tyrosine kinase inhibitor;
² mAb, monoclonal antibody

Cells plated in culture media containing 5% fetal calf serum at a density of 2.6×10⁵ (SKOV3) or 6×10⁵ (A2780) in 60 mm dishes (Corning, Corning, N.Y.) were treated with FASN inhibitor C75, with small-molecule ErbB RTK inhibitors (RTKIs), with the MEK1/2 antagonist U0126 or with the PI3K inhibitor LY294002 for the indicated times and at various concentrations. In some experiments cells were stimulated for 5 or 10 min with 100 ng/ml EGF (Sigma) before lysis in RIPA buffer (Grunt et al., Biochem Biophys Res Commun 329:1253-1259, 2005). Proteins (20 μg/lane) were subjected to SDS-PAGE, blotted onto PVDF membranes and immunostained (see Grunt, ibid) using the following antibodies: 2 μg/ml goat anti-pEGFR(Tyr1173) (sc-12351), 1 μg/ml rabbit anti-ErbB2 (C-18) (sc-284), 1 μg/ml rabbit anti-pErbB2(Tyr1248) (sc-12352-R) (all from Santa Cruz Biotechnology Inc., Santa Cruz, Calif.), rabbit anti-EGFR 1:1,000 (2232), rabbit anti-AKT 1:1,000 (9272), rabbit anti-pAKT(Ser473) 1:1,000 (9271), rabbit anti-pAKT(Thr308) 1:1,000 (9275), rabbit anti-pERK1/2(Thr202/Tyr204) 1:1,000 (9101) (all from Cell Signaling Technology), rabbit anti-ERK1/2 1:3,000 (06-182, Upstate Biotechnology Inc., Lake Placid, N.Y.), 0.5 μg/ml mouse monoclonal anti-FASN (610963, BD Biosciences, San Jose, Calif.), or 0.67 μg/ml goat anti-human actin (1-19, sc-1616, Santa Cruz Biotechnology). Secondary antibodies were peroxidase-tagged donkey-anti-rabbit IgG (V795A, Promega, Madison, Wis.) or donkey-anti-goat IgG (sc-2020, Santa Cruz Biotechnology) at 1:15,000, respectively, or chicken anti-mouse IgG (sc-2954, Santa Cruz Biotechnology) at 1:10,000. Detection was performed by enhanced chemiluminescence.

Cells were plated at 0.5−5×10³/well in 96-well plates. After adhesion, media ±drugs alone or in combination were added. Cell numbers were estimated after 3-5 days by using a colorimetric formazan dye assay (Biomedica, Vienna, Austria). Results (mean±SD, n=3) were expressed in percent of untreated (solvent) control. Statistical analysis of data from drug combination treatment experiments was performed using one-factorial ANOVA followed by Scheffe-test.

RNA was extracted from the cells using TRI-REAGENT™ (Molecular Research Center, Cincinnati, Ohio) and subjected to reverse transcription—polymerase chain reaction (“RT-PCR”) using the TITAN ONE TUBE RT-PCR KIT™ system (Hoffmann-La Roche). Primers were: EGFR: 5′-TTCAAGACCTGGCCCAGTGCATCC-3′ (SEQ ID NO:1) (forward, E1S) 5′-AGCAACAACCCTGCCCTGTGCAAC-3′ (SEQ ID NO:2) (reverse, E1A); ErbB-2: 5′-CACTTCAACCACAGTGGCAT-3′ (SEQ ID NO:3) (forward, E2S2), 5′-ATTCACATACTCCCTGGGGA-3′ (SEQ ID NO:4) (reverse, E2A2); GAPDH: 5′-GAGAACGGGAAGCTTGTCAT-3′ (SEQ ID NO:5) (forward, GAPDH3S), 5′-TTCAGCTCAGGGATGACCTT-3′ (SEQ ID NO:6) (reverse, GAPDH3A). Conditions for reverse transcription and amplification of the EGFR coding sequence were: 50° C., 30 minutes; 94° C., 2 minutes; 10 cycles (94° C., 30 seconds; 64.9° C., 30 seconds; 68° C., 90 seconds); 30 cycles (94° C., 30 seconds; 64.9° C., 30 seconds; 68° C., 90 seconds +5 seconds autoextension) and 68° C., 7 minutes. Conditions for ErbB2 and GAPDH were: 50° C., 30 minutes; 94° C., 2 minutes; 10 cycles (94° C., 30 seconds; 51° C., 30 seconds; 68° C., 45 seconds); 25 cycles (94° C., 30 seconds; 51° C., 30 seconds; 68° C., 45 seconds +5 seconds autoextension) and 68° C., 7 minutes. DNA products had the following sizes: EGFR: 1452 bp, ErbB2: 900 bp, GAPDH: 488 bp. Fragments were electrophoresed and stained with ethidium bromide.

Quantitative RT-PCR (“qRT-PCR”) was performed in the ABI PRISM 7000™ Sequence Detector (Applied Biosystems, Foster City, Calif.) using human EGFR, ErbB2, and FASN TaqMan® Gene Expression Assay systems (Applied Biosystems; Assay IDs: Hs00193306_m1, Hs00170433_m1, Hs00188012_m1) according to the manufacturer's instructions. β-actin and GAPDH (VIC™ and FAM labeled pre-developed TaqMan® Assay reagents, Applied Biosystems) were used as endogenous controls. EGFR, ErbB2, and FASN mRNA expression were calculated as described previously (Grunt, ibid).

Logarithmically growing SKOV3 ovarian cancer cells were transfected with 25 nM siRNA oligonucleotides specific for EGFR or ErbB2 using siRNA transfection reagents obtained from Dharmacon (Lafayette, Colo.). The procedure was performed according to the manufacturer's instructions, with slight modifications. For example, siRNA uptake into SKOV3 cells was performed with DHARMAFECT™ 3 (0.9375 μl/ml). For detection of EGFR and ErbB2 mRNA expression, 10,000 cells/well were plated in α-MEM containing 6.25% fetal calf serum and 2 mM glutamine (Gibco) in 96-well plates.

25 nM siRNAs (diluted in α-MEM containing 2 mM glutamine) were added to freshly plated floating cells and incubated for 72 hours. Then cells were lysed and subjected to quantitative RNA determination using branched DNA assay technology. To this end, aliquots of the lysates equal to approximately 600 cells/well were transferred into individual wells of plates of the QUANTIGENE 2.0™ REAGENT SYSTEM (Panomics, Fremont, Calif.). Detection of EGFR and ErbB2 mRNAs was then performed according to the manufacturer's protocol (see also Warrior et al., J. Biomol. Screen., 5(5):343-52, 2000). Luminescence signals directly correlating with mRNA levels were measured with a multidetection plate reader (FLUOSTAR OPTIMA™, BMG Labtech GmbH, Offenburg, Germany). For detection of protein expression, 3×10⁵ cells were plated into 35 mm cell culture dishes (Corning) using the same medium as above, exposed to 25 nM siRNAs in DHARMAFECT™ 3 and incubated for 72 hours. Cells were then lysed in RIPA buffer and processed for regular Western blotting as described above.

### Expression of EGFR, ErbB2 and FASN Proteins in A2780 and SKOV3 Ovarian Carcinoma Cells

Baseline levels of EGFR, ErbB2 and FASN protein expression were determined in A2780 and SKOV3 ovarian carcinoma cells by Western blot analysis using actin as a control. A2780 and SKOV3 ovarian cancer cells revealed opposite expression profiles for EGFR, ErbB2 and FASN proteins, whereby SKOV3 cells expressed high levels of EGFR and ErbB2 and moderate amounts of FASN, and A2780 contained much less EGFR and ErbB2, but markedly more FASN protein than SKOV3 cells.

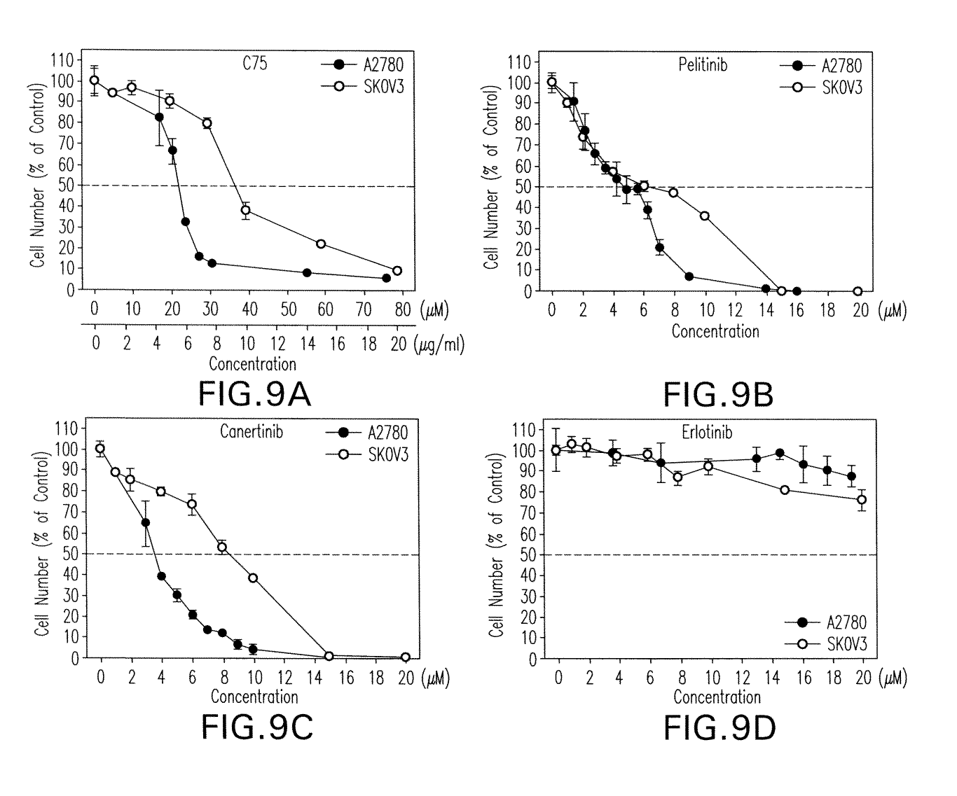

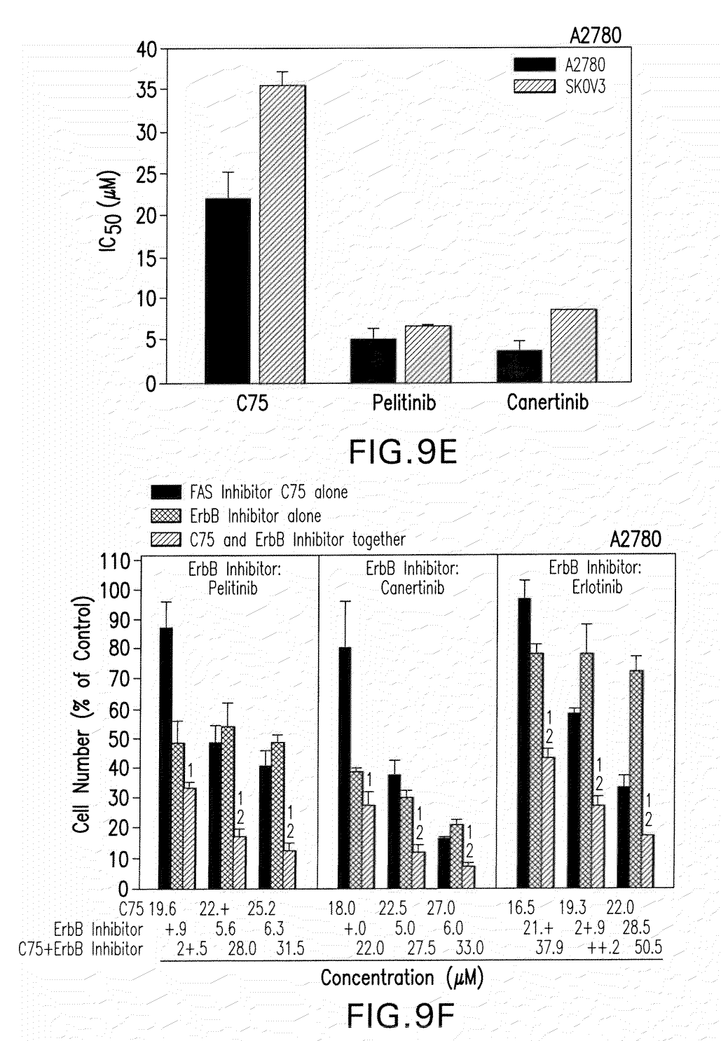

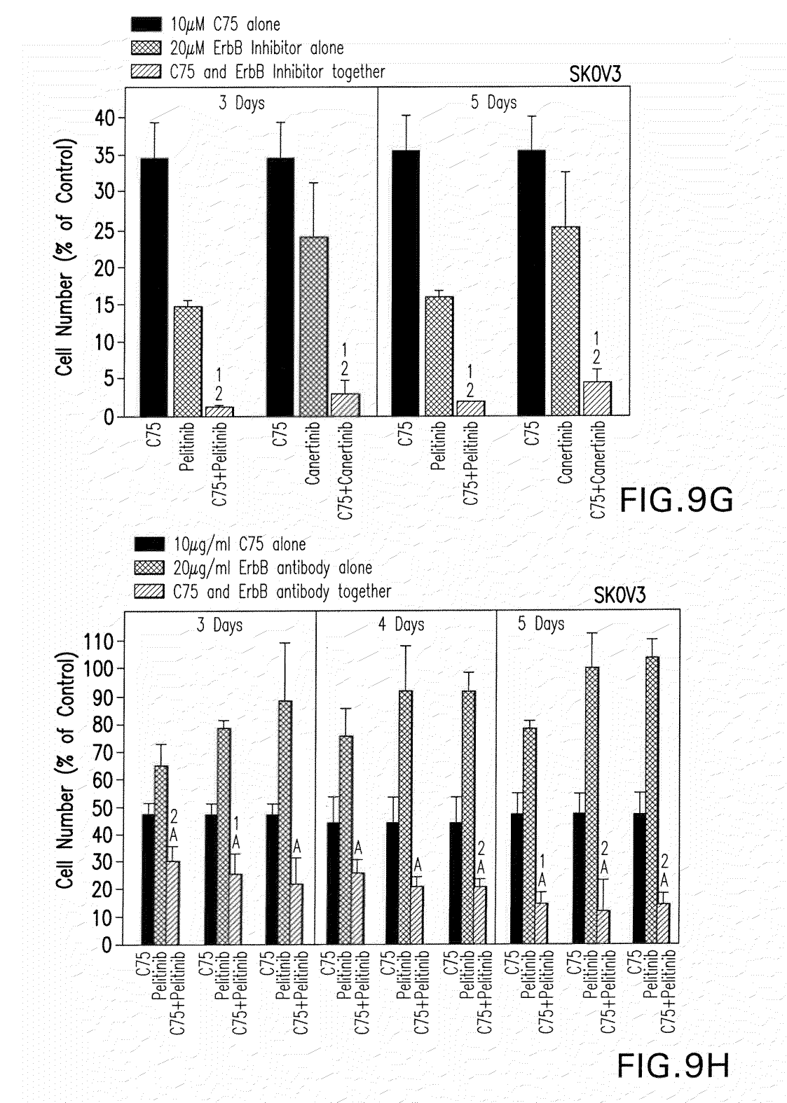

In vitro growth experiments using the MTT assay revealed growth inhibitory activity for the FASN antagonist C75 and for the ErbB inhibitors pelitinib (EKB-569) and canertinib (CI-1033) in the ovarian adenocarcinoma cell lines A2780 and SKOV3, whereas erlotinib (OSI-774, Tarceva) does not significantly inhibit growth of these cell lines (FIG. 9a). Yellow MTT (3-(4,5-Dimethylthiazol-2-yl)-2,5-diphenyltetrazolium bromide, a tetrazole) is reduced to purple formazan in the mitochondria of living cells (see Mosmann, T., J. Immunol. Methods, 65(1-2): 55-63, 1983).

The estimated IC₅₀ values depicted in FIG. 9b demonstrate that A2780 cells are slightly more drug-sensitive than SKOV3 cells. Interestingly, co-exposure of A2780 cells to C75 and ErbB inhibitors causes growth inhibition even when the single drugs are not significantly active. Therefore, C75 and the ErbB inhibitors pelitinib, canertinib and erlotinib appear to cooperate for in vitro growth inhibition of A2780 cells (FIG. 9c). Likewise, antiproliferative cooperation between the FASN antagonist and the ErbB RTKIs was also seen in SKOV3 cells (FIG. 9d). The effects of the EGFR-blocking antibodies cetuximab and matuzumab, and of the ErbB2-neutralizing antibody trastuzumab was also examined in these cells. At a concentration of 20 μg/ml, trastuzumab causes >20%, cetuximab <20%, and matuzumab ˜10% growth inhibition. Surprisingly, when 10 μg/ml C75, which on its own inhibits in vitro growth of SKOV3 cells by 50-55%, is added to the antibodies, growth inhibition is significantly enhanced to >70% (FIG. 9e). These data clearly demonstrate that A2780 and SKOV3 cell growth is sensitive to inhibition of FASN and ErbB pathways, and blockade of both systems synergistically causes cooperative growth control in these ovarian cancer cells.

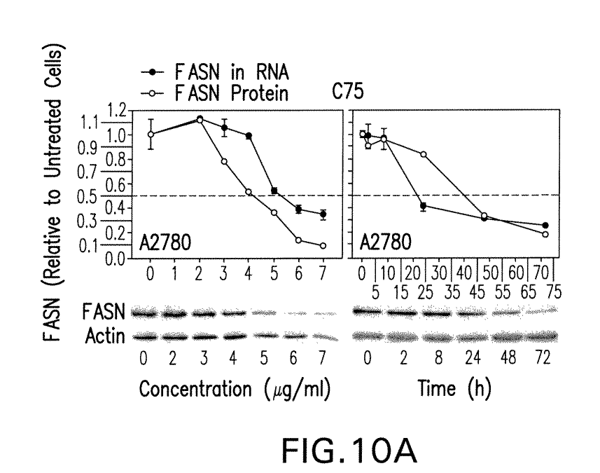

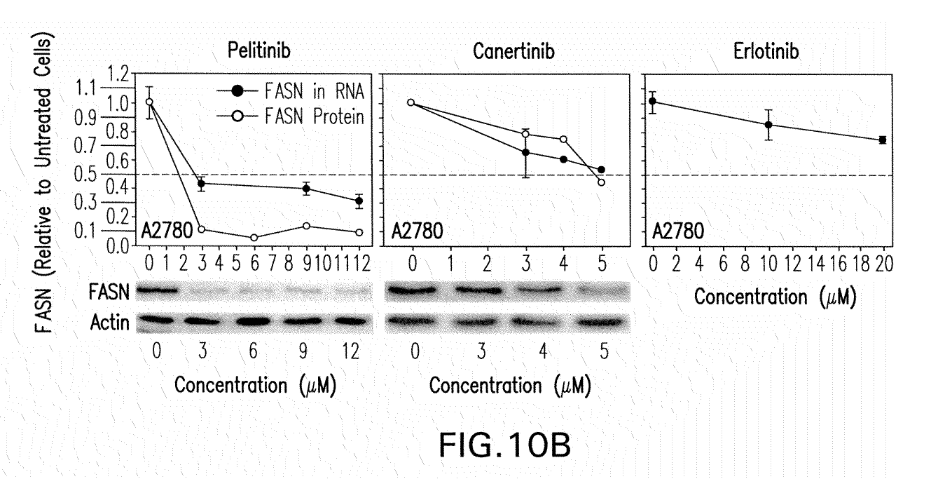

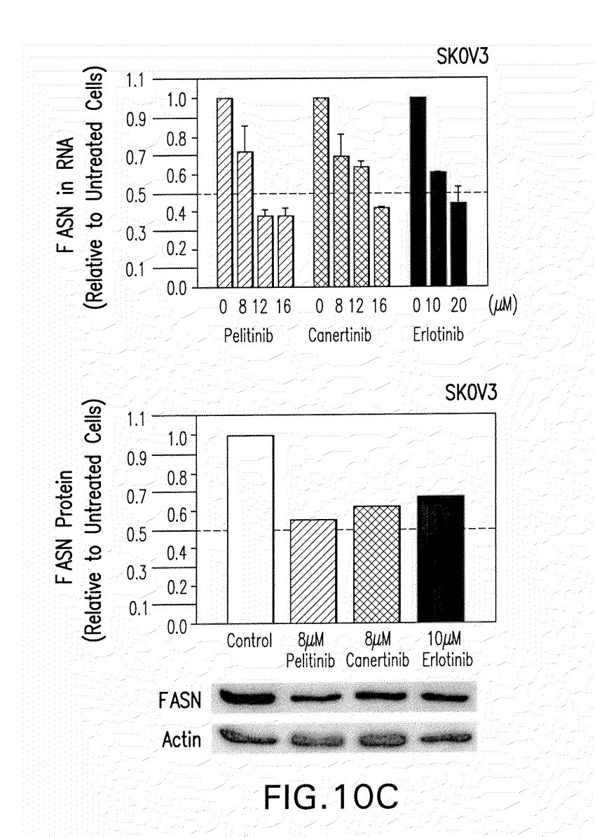

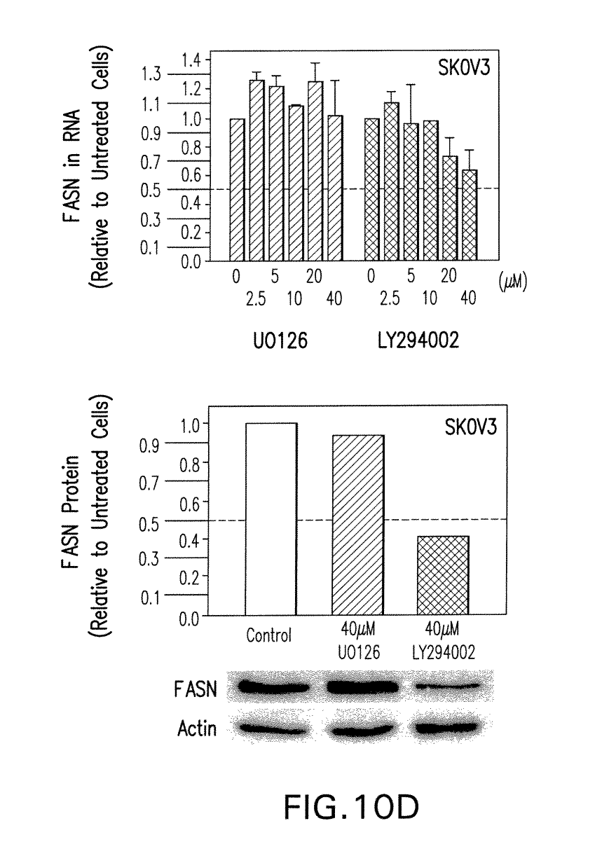

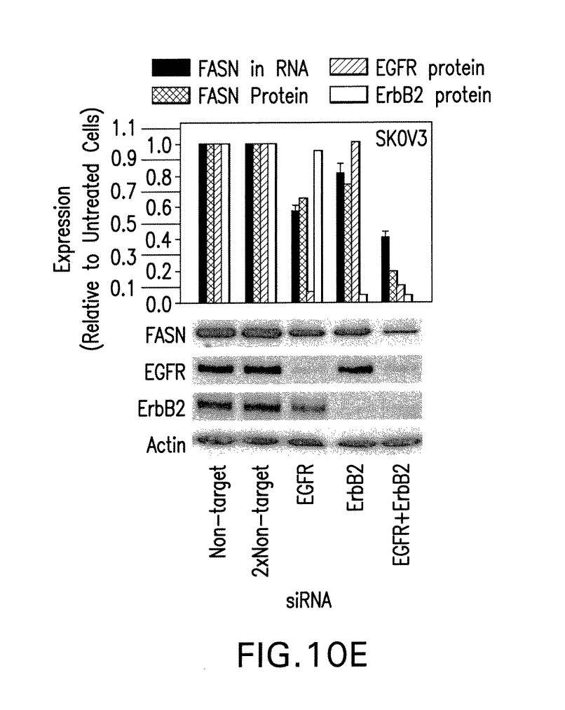

qRT-PCR revealed that C75 down-regulates FASN mRNA expression in a dose- and time-dependent manner in A2780 cells with an IC₅₀ value after 3 days of 5.25 μg/ml and an IT₅₀ (time of exposure after which a 50% inhibition is obtained) at 7 μg/ml of 21.5 hours. This corresponds with a concordant down-regulation of FASN protein (IC₅₀=4.15 μg/ml), although the dynamics are slower (IT₅₀=40 hours) (FIG. 10a). Surprisingly, drug-mediated inhibition of ErbB activity also caused marked down-regulation of FASN mRNA and protein steady-state levels. For instance, within three days pelitinib abrogated FASN mRNA and protein expression in A2780 cells at doses as low as 3 μM. This effect was also seen with the other two ErbB inhibitors, canertinib and erlotinib (FIG. 10b). Moreover, comparative experiments using SKOV3 cells revealed that all three ErbB targeting drugs dose-dependently reduce FASN mRNA transcript levels in these cells as well, which again is accompanied by reduced protein expression (FIG. 10c). Protein levels were reduced to a greater degree than the mRNA levels.

To characterize the relative contribution of the MAPK and PI3K/AKT pathways to ErbB/FASN cross-regulation, SKOV3 cells were exposed to the PI3K inhibitor LY294002 or the MAPK kinase 1/2 (MEK1/2) inhibitor U0126 and the FASN protein and mRNA levels were determined. In contrast to U0126, which does not affect FASN mRNA and protein levels, LY294002 reduces both FASN mRNA and protein (FIG. 10d). As seen with the ErbB RTKIs, protein levels were reduced to a greater degree than the mRNA levels. (FIG. 10d). These data suggest that ErbB-dependent control of FASN expression is mediated via the PI3K/AKT pathway and that (post)translational mechanisms might significantly contribute to the overall effect.

ErbB-dependent modulation of FASN gene expression was further examined by RNA interference. Transfection of siRNAs directed against EGFR or ErbB2 into SKOV3 cells selectively silences the intended target gene, respectively, while leaving the non-targeted ErbB receptor unaffected. However, both EGFR and ErbB2 siRNAs markedly compromise FASN mRNA and protein expression. Surprisingly, FASN silencing is even much more pronounced, when both siRNAs are simultaneously transfected. Each siRNA alone reduces the FASN level by 30-40%, whereas both siRNAs together cooperatively attenuate FASN expression by at least 80%. (FIG. 10e). siRNAs directed to EGFR are depicted in the sequence listing, the entirety of which forms a part of the instant disclosure, as SEQ ID NO:7-SEQ ID NO:14. siRNAs directed to ErbB-2 are depicted in the sequence listing as SEQ ID NO:15-SEQ ID NO:22.

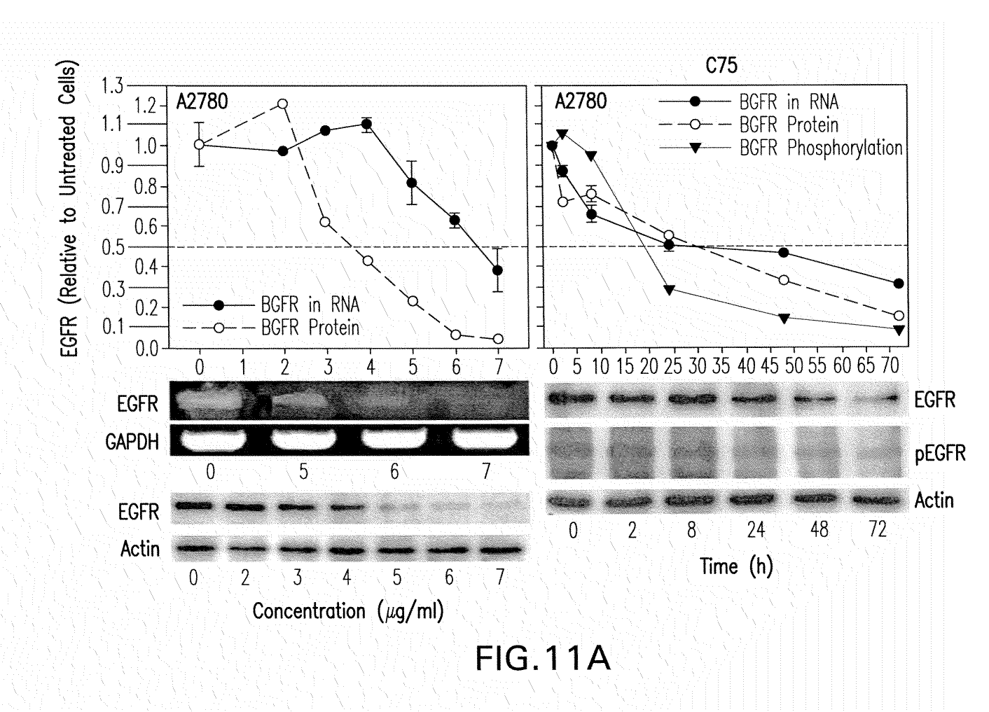

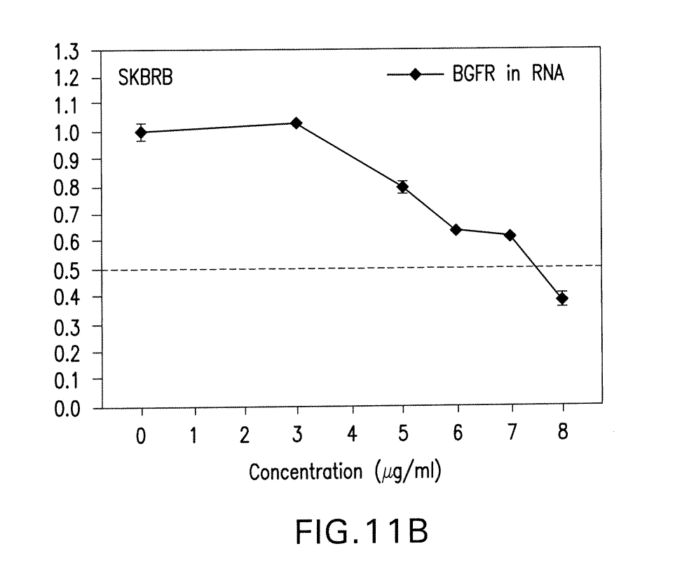

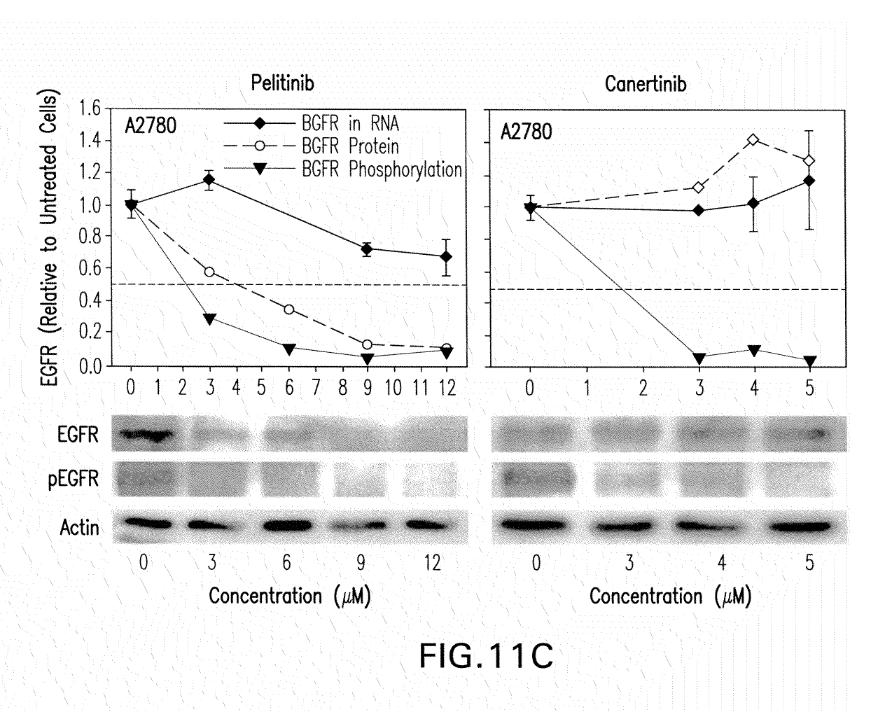

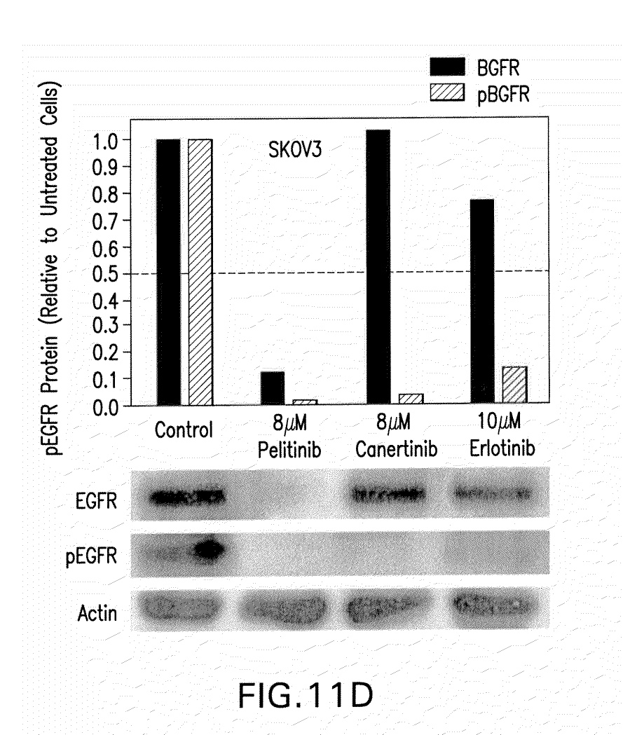

The FASN inhibitor C75 dose- and time-dependently down-regulates EGFR mRNA in A2780 cells with an IC₅₀ value after 3 days of 6.5 μg/ml and an IT₅₀ value at 7 μg/ml at about 29 hours as demonstrated by RT-PCR and qRT-PCR (FIG. 11a). Correspondingly, a similar effect was obtained in SKBR3 breast cancer cells (IC₅₀ 7.5 μg/ml). This correlated with reduced levels of EGFR protein and EGFR tyrosine phosphorylation as demonstrated by Western blot analysis of C75-treated A2780 cells (FIG. 11a, lower panels). Although the IC₅₀ for EGFR protein is somewhat lower than that for EGFR mRNA (3.6 μg/ml vs. 6.5 μg/ml, respectively), the IT₅₀ values for EGFR mRNA (29 hours), EGFR protein (29 hours), and EGFR phosphorylation (20 hours) are in the same range. Thus, C75-mediated blockade of FASN function reduces EGFR expression and subsequent activity most likely at the mRNA level.

The effect of ErbB targeting drugs on EGFR transcript and protein levels, and on EGFR phosphorylation was examined. The irreversible EGFR/ErbB2-specific RTKI pelitinib completely abrogates EGFR tyrosine phosphorylation, which entails dose-dependent down-regulation of the corresponding total protein and transcript levels (FIG. 11b, left panels). In contrast, canertinib, the other irreversible ErbB RTKI tested, although known to efficiently silence EGFR function, does not diminish EGFR protein and mRNA (FIG. 11b, right panels). This indicates that pharmacological blockade of EGFR does not always induce the same molecular effects on the target irrespective of the individual ErbB RTKI used. Correspondingly, similar results were obtained when SKOV3 cells, which overexpress EGFR, were treated with pelitinib, canertinib or erlotinib (FIG. 11c).

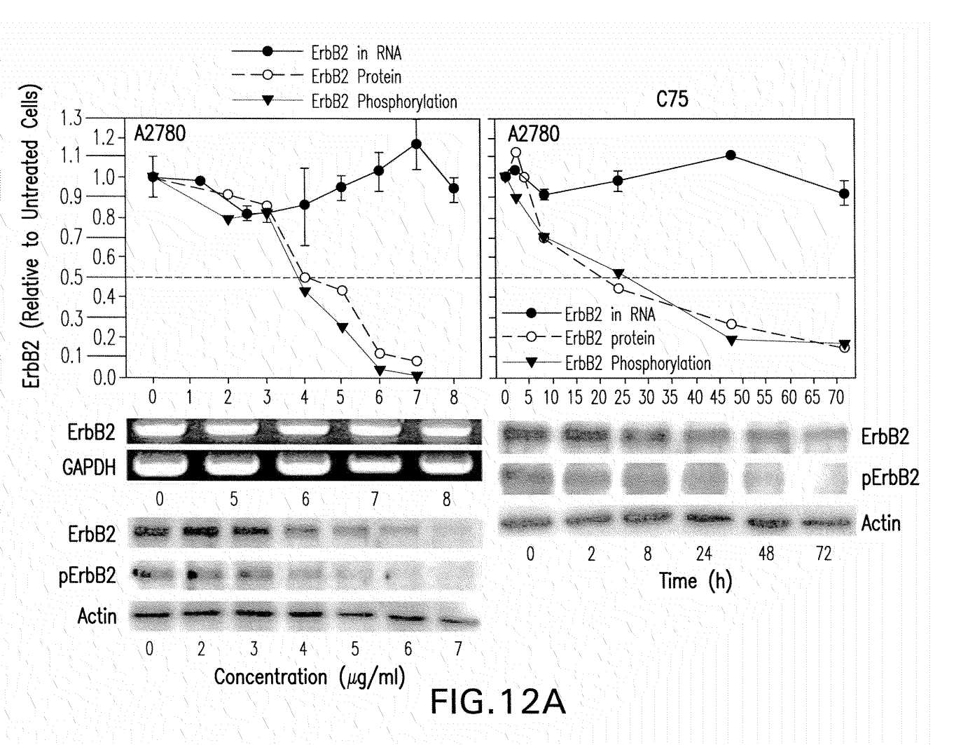

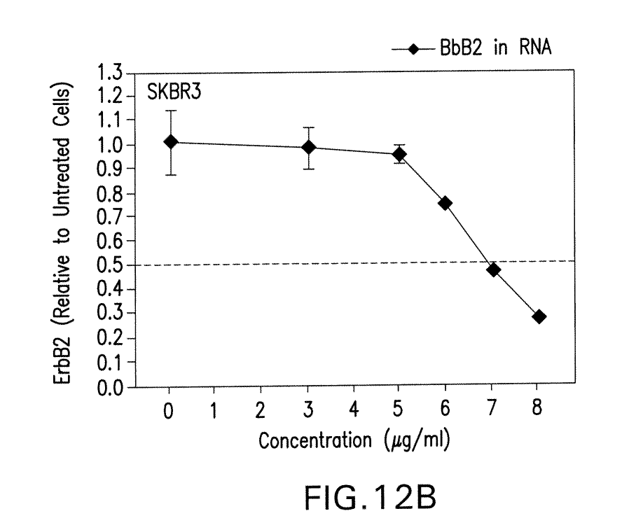

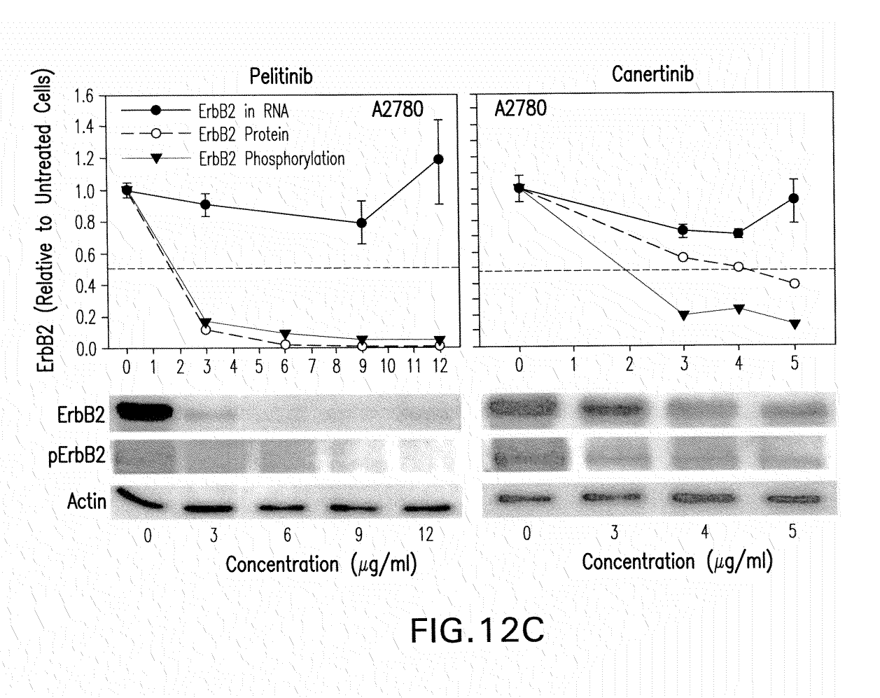

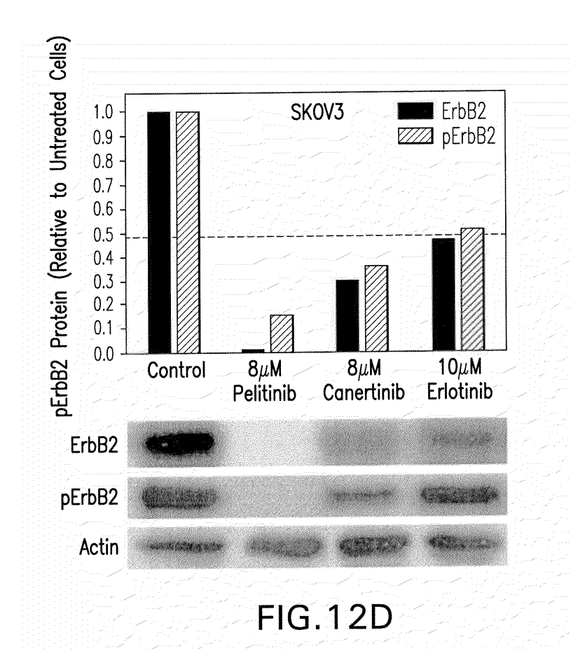

Unlike EGFR, ErbB2 mRNA expression is not affected by C75 in A2780 ovarian cancer cells. Nevertheless, C75 downregulates total and phosphorylated ErbB2 protein in these cells. In SKBR3 breast cancer cells, a dose-dependent decline of ErbB2 mRNA was seen after exposure to C75 (FIG. 12a) corroborating previous reports from Menendez et al., PNAS, 101:10715-20, 2004. Strikingly, the corresponding quantitative data for ErbB2 and pErbB2 in A2780 cells were almost identical (FIG. 12a) indicating that C75-dependent blockade of FASN reduced ErbB2 expression and function in these cells via (post-)translational mechanisms, whereas in SKBR3 cells transcriptional repression appears to be a mechanism of action.

Like EGFR, ErbB2 protein phosphorylation and expression are abrogated by pelitinib, whereas ErbB2 transcript levels do not appear to be affected. In comparison, canertinib strongly diminishes pErbB2 and slightly attenuates ErbB2, but fails to down-regulate ErbB2 mRNA. Corresponding results were obtained when SKOV3 cells were exposed to pelitinib, canertinib or erlotinib (FIG. 12c).

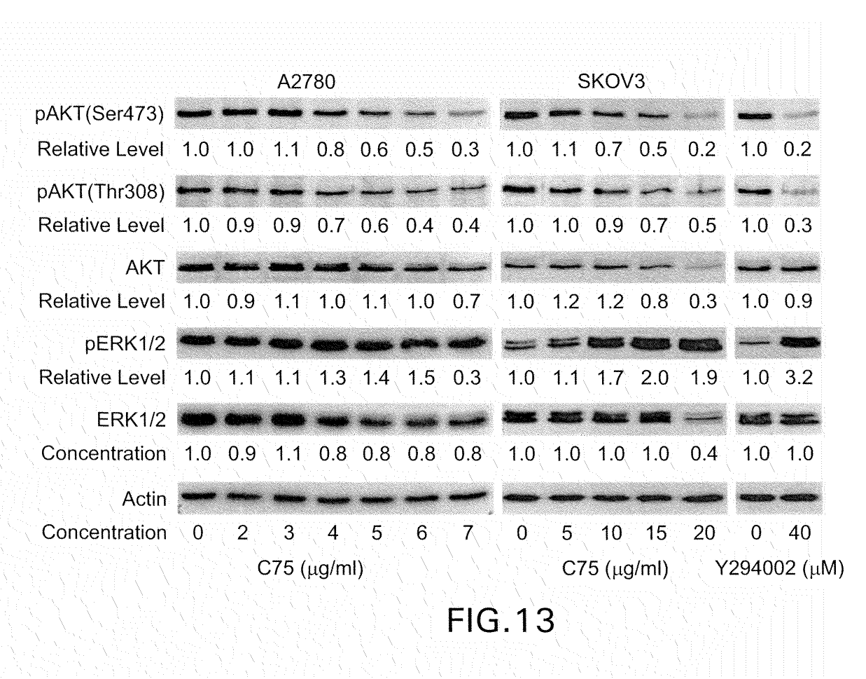

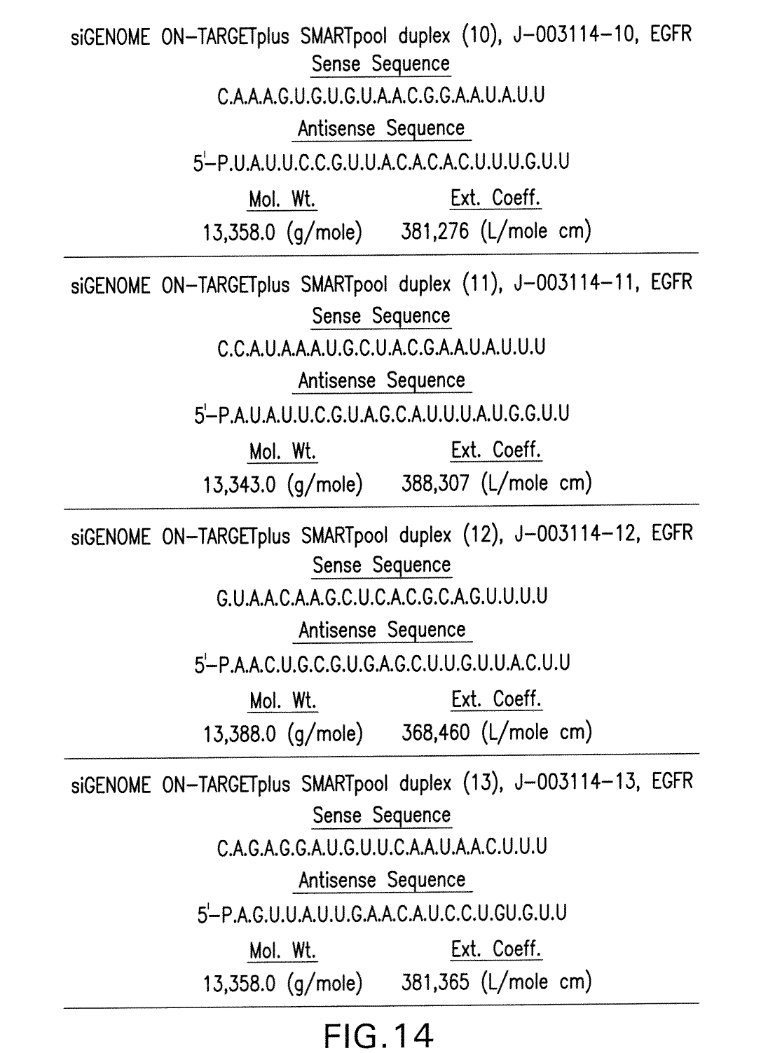

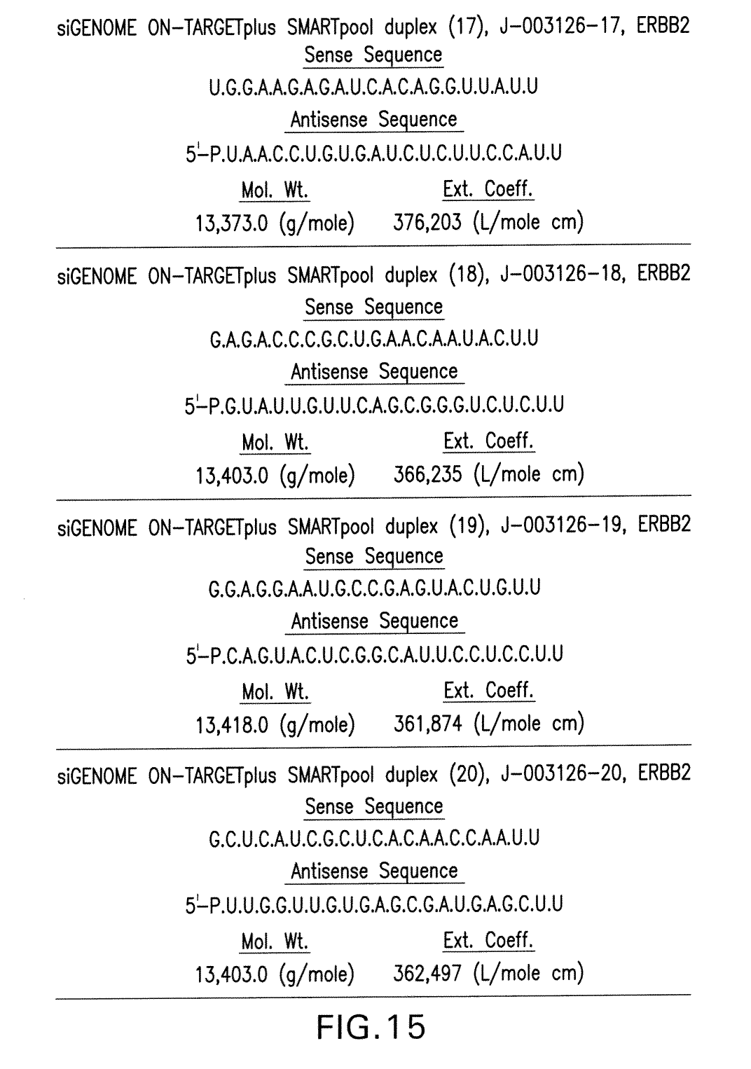

After having demonstrated that inhibition of FASN compromises EGFR and ErbB2 expression in ovarian cancer, the effects of C75 on the phosphorylation and expression of AKT and ERK1/2, the crucial mediators of two major ErbB downstream cascades, were examined (FIG. 13). Interestingly, C75 dose-dependently inhibits phosphorylation of AKT at the two functionally relevant amino acid positions Ser473 and Thr308 in both ovarian cancer cell lines tested. Moreover, at high concentrations C75 also slightly affects total AKT protein level. In contrast, pERK1/2 (phosphoERK1/2) levels dose-dependently increase upon C75 administration in both A2780 and SKOV3, even though total ERK1/2 levels concurrently decline. In comparison, PI3K inhibitor (LY294002)-mediated blockade of AKT is correspondingly correlated with a compensatory hyperactivation of ERK1/2. However, unlike C75, LY294002 does not lower total AKT and ERK1/2 levels (FIG. 13).

What is claimed is:

1. A pharmaceutical composition comprising: a receptor tyrosine kinase inhibitor, or a pharmaceutically acceptable salt thereof, and a fatty acid synthase inhibitor.

2. The pharmaceutical composition of claim 1, wherein the receptor tyrosine kinase inhibitor is an epidermal growth factor receptor inhibitor.

3. The pharmaceutical composition of claim 1, wherein the receptor tyrosine kinase inhibitor is an epidermal growth factor receptor inhibitor, ErbB.

4. The pharmaceutical composition of claim 1, wherein the receptor tyrosine kinase inhibitor is a cyanoquinoline, or a pharmaceutically acceptable salt thereof, and the fatty acid synthase inhibitor is tetrahydro-3-methylene-2-oxo-5-n-octyl-4-furancarboxylic acid.

5. The pharmaceutical composition of claim 1, wherein the receptor tyrosine kinase inhibitor is 4-Dimethylamino-but-2-enoic acid [4-(3-chloro-4-fluoro-phenylamino)-3-cyano-7-ethoxy-quinolin-6-yl]-amide, or a pharmaceutically acceptable salt thereof, and the fatty acid synthase inhibitor is tetrahydro-3-methylene-2-oxo-5-n-octyl-4-furancarboxylic acid.

6. The pharmaceutical composition of claim 1, wherein the receptor tyrosine kinase inhibitor is (E)-N-{4-[3-chloro-4-(2-pyridinyl methoxy) anilino]-3-cyano-7-ethoxy-6-quinolinyl}-4-(dimethylamino)-2-butenamide, or a pharmaceutically acceptable salt thereof, and the fatty acid synthase inhibitor is tetrahydro-3-methylene-2-oxo-5-n-octyl-4-furancarboxylic acid.

7. The pharmaceutical composition of claim 1, wherein the receptor tyrosine kinase inhibitor is (E)-N-(4-{3-chloro-4-[(3-fluorobenzyl)oxy]anilino}-3-cyano-7-ethoxy-6-quinolinyl)-4-(dimethylamino)-2-butenamide, or a pharmaceutically acceptable salt thereof, and the fatty acid synthase inhibitor is tetrahydro-3-methylene-2-oxo-5-n-octyl-4-furancarboxylic acid.

8. The pharmaceutical composition of claim 1, wherein the receptor tyrosine kinase inhibitor is 4-(2,4-dichloro-5-methoxyanilino)-7-{5-[(4-methyl-1-piperazinyl)methyl]-2-pyridinyl}-3-carbonitrile, or a pharmaceutically acceptable salt thereof, and the fatty acid synthase inhibitor is tetrahydro-3-methylene-2-oxo-5-n-octyl-4-furancarboxylic acid.

9. A pharmaceutical composition according to claim 1, further comprising a pharmaceutically acceptable excipient or carrier.

10. A method for treating cancer by administering to a patient a pharmaceutically effective amount of a pharmaceutical composition as defined in any one of claim 1 and a pharmaceutically acceptable carrier.

11. The method of claim 10, wherein the cancer is ovarian cancer.

12. A method for treating ovarian cancer comprising: administering to a patient a pharmaceutically effective amount of 4-Dimethylamino-but-2-enoic acid [4-(3-chloro-4-fluoro-phenylamino)-3-cyano-7-ethoxy-quinolin-6-yl]-amide, or a pharmaceutically acceptable salt thereof, and tetrahydro-3-methylene-2-oxo-5-n-octyl-4-furancarboxylic acid.

13. A method for reducing activity in a cell, said activity selected from one or more of ErbB-2 activity, fatty acid synthase activity and epidermal growth factor receptor activity, comprising contacting the cell with a compound that inhibits fatty acid synthase activity.

14. The method of claim 13 further comprising contacting the cell with an inhibitor of ErbB-2.

15. The method of claim 14 wherein the ErbB-2 activity is the expression of an ErbB-2 mRNA.

16. The method of claim 14 wherein the ErbB-2 activity is the production of an ErbB-2 protein.

17. The method of claim 14 wherein the ErbB-2 activity is the phosphorylation of an ErbB-2 protein.

18. The method of claim 13 wherein compound that inhibits fatty acid synthase activity is a small molecule.

19. The method of claim 13 wherein the compound that inhibits fatty acid synthase activity is a protein.

20. The method of claim 19 wherein the protein is a polypeptide comprising a complementarity determining region that recognizes a fatty acid synthase epitope.

21. The method of claim 20 wherein the protein is an antibody that binds to fatty acid synthase.

22. The method of claim 13 wherein the compound that inhibits fatty acid synthase activity is a polynucleotide.

23. The method of claim 22 wherein the polynucleotide is a siRNA.

24. The method of claim 23 wherein the polynucleotide is selected from the group comprising SEQ ID NO:15-SEQ ID NO:22.

25. The method of claim 23 wherein the polynucleotide is selected from the group comprising SEQ ID NO:7-SEQ ID NO:14.

26. A method for inhibiting the proliferation of an ovarian cell comprising contacting the cell with:
(a) a combination comprising a compound that inhibits fatty acid synthase activity and a compound that inhibits ErbB-2 activity; or
(b) a combination comprising a compound that inhibits fatty acid synthase activity and a compound that inhibits epidermal growth factor receptor activity,
wherein the rate of cell division for the cell is less than 50% of the rate of cell division for the cell in the absence combination (a) and combination (b).

27. The method of claim 13 wherein the cell is a cancer cell.
